(19) United States
(12) Patent Application Publication
(10) Pub. No.: US 2026/0174715 A1
(43) Pub. Date: Jun. 25, 2026

(54) FORMULATIONS FOR INJECTABLE COMBINATION PRODUCT CONTAINING ACETAMINOPHEN AND IBUPROFEN

(71) Applicant: Hikma Pharmaceuticals USA Inc., Berkeley Heights, NJ (US)

(72) Inventor: Timothy Owen, Bedford, OH (US)

(21) Appl. No.: 19/001,066

(22) Filed: Dec. 24, 2024

(51) Int. Cl. A61K 31/192; A61J 1/14; A61K 31/167; A61K 47/02; A61K 47/18

(52) U.S. Cl. CPC A61K 31/192; A61J 1/1468; A61K 31/167; A61K 47/02; A61K 47/183

(57) ABSTRACT

The present disclosure relates to liquid formulations containing acetaminophen and ibuprofen. In particular, the present disclosure relates to liquid formulations comprising acetaminophen, ibuprofen, and a sulfite compound. Such liquid formulations are suitable for parenteral (e.g., intravenous) administration. The present disclosure also relates to terminally-sterilized acetaminophen/ibuprofen liquid formulations. The present disclosure also relates to methods of treating patients by administration of such formulations and polymeric infusion containers, including flexible polymeric infusion containers, containing such formulations.

### CROSS-REFERENCE TO RELATED APPLICATIONS

N/A.

### TECHNICAL FIELD

The present invention relates to formulations for an acetaminophen/ibuprofen combination product, methods for preparing such formulations, methods for using such formulations, and polymeric infusion containers, such as flexible polymeric infusion containers, filled with such formulations.

### BACKGROUND

Acetaminophen is a non-opiate, non-salicylate analgesic and antipyretic drug. Ibuprofen is a nonsteroidal anti-inflammatory drug (NSAID) that possesses analgesic and antipyretic activities.

There is a demand in the field for products that combine acetaminophen and ibuprofen, especially in a form suitable for intravenous injection. One such commercial product is a vial product, which is currently available as COMBOGESIC® IV (acetaminophen and ibuprofen) in the United States and under the trade name of MAXIGESIC® IV outside of the United States. COMBOGESIC® IV is an intravenous, opioid-free pain relief medicine that is a combination of 1,000 mg of acetaminophen and 300 mg of ibuprofen provided in a single-dose borosilicate glass vial. Each single-dose 100 mL vial contains 1,000 mg of acetaminophen and 300 mg of ibuprofen base (equivalent to 385 mg of ibuprofen sodium dihydrate), 25 mg of Cysteine hydrochloride monohydrate, 13 mg of Disodium phosphate dihydrate, 3285 mg of Mannitol, Hydrochloric acid (for pH adjustment), Sodium hydroxide (for pH adjustment), and Water For Injection.

There is an unmet demand in the field for an acetaminophen/ibuprofen combination product that is supplied in a polymeric infusion container, including flexible polymeric infusion containers. In addition, there is an unmet demand for ready-to-administer injectable formulations of an acetaminophen/ibuprofen combination product that offer long-term storage stability and can be terminally sterilized after filling, particularly after filling into a flexible polymeric infusion container.

### SUMMARY OF THE INVENTION

This disclosure is directed to ready-to-administer parenteral liquid formulations comprising acetaminophen and ibuprofen or a pharmaceutically acceptable salt thereof such as ibuprofen sodium and, in particular, ibuprofen sodium dihydrate. The liquid formulations further comprise an antioxidant, such as a sulfite compound. The liquid formulations further comprise a buffering agent having a buffering capacity near physiological pH, such as between pH 6 and pH 10, or between pH 6 and pH 8, preferably between pH 6.0 and pH 7.5. Exemplary buffering agents include sodium bicarbonate, sodium citrate, or an amino acid such as glycine or L-histidine. In certain embodiments, these liquid formulations have been terminally sterilized in a flexible polymeric infusion container and are stable upon long-term storage. As disclosed herein, liquid formulations terminally sterilized in flexible polymeric infusion containers are stable when stored under long-term storage conditions for up to six (6) months.

As described herein, applicant discovered that the existing glass vial formulation containing, inter alia, L-cysteine hydrochloride monohydrate and disodium phosphate dihydrate, is not stable in a flexible polymeric infusion container upon terminal sterilization. One advantageous property of the formulations described herein is their capacity to withstand terminal sterilization in a flexible polymeric infusion container.

In one aspect, this disclosure provides a terminally sterilized, injectable acetaminophen and ibuprofen liquid formulation packaged in a ready-to-administer (RTA) intravenous (IV) flexible polymeric container. In certain embodiments, the concentration of acetaminophen is 10 mg/mL, such as 1000 mg in 100 mL. In certain embodiments, the concentration of ibuprofen is 3 mg/mL, such as 300 mg in 100 mL; the ibuprofen may be present as pharmaceutically acceptable salt such as ibuprofen sodium and, more particularly, ibuprofen sodium dihydrate (i.e., 300 mg of ibuprofen equivalent to 385 mg of ibuprofen sodium dihydrate).

In one aspect, this disclosure provides a parenteral liquid formulation comprising acetaminophen, ibuprofen, and an antioxidant. The liquid formulation may further comprise an isotonic agent, such as mannitol. The liquid formulation may further comprise a buffering agent having a buffering capacity near physiological pH, such as between pH 6 and pH 10, or between pH 6 and pH 8, preferably between pH 6.0 and pH 7.5. Exemplary buffering agents include sodium bicarbonate, sodium citrate, glycine, or L-histidine. The antioxidant is a sulfite compound, such as sodium metabisulfite, sodium sulfite, and sodium bisulfite. In certain embodiments, the sulfite compound is present in the liquid formulation in an amount from 0.0625 to 1.0 mg/ml at the time of preparation of the liquid formulation. The parenteral liquid formulation is capable of withstanding terminal sterilization in a polymeric infusion container, such as a flexible polymeric infusion container. In certain embodiments, the flexible polymeric infusion container comprises a polyolefin-based film, such as PolyCine Advanced PolyPropylene (“APP”) 114. In certain embodiments, the parenteral liquid formulation has an American Public Health Association (APHA) color of 52 or less following heat-sterilization (e.g., by an autoclave) in a flexible polymeric infusion container with an F₀ value of 15. In certain embodiments, the parenteral liquid formulation has an APHA color of 128 or less following heat-sterilization (e.g., by an autoclave) in a flexible polymeric infusion container with an F₀ value of 15 and subsequent storage under long-term storage conditions for up to 1, 2, 3, 6, 9, 12, or 24 months.

In one aspect, this disclosure also provides a parenteral liquid formulation comprising acetaminophen, ibuprofen, and a sulfite compound as an antioxidant, wherein the liquid formulation has been terminally sterilized (e.g., by an autoclave) in a flexible polymeric infusion container, wherein terminal sterilization is characterized by an F₀ value from 5 to 15; and wherein the liquid formulation has an APHA color of (a) 52 or less following heat-sterilization and/or (b) 128 or less following heat-sterilization and subsequent storage under long-term storage conditions for up to 1, 2, 3, 6, 9, 12, or 24 months. In certain embodiments, the sulfite compound is selected from the group consisting of sodium metabisulfite, sodium sulfite, and sodium bisulfite. In certain embodiments, the sulfite compound is present in the liquid formulation in an amount from 0.0625 to 1.0 mg/ml at the time of preparation of the liquid formulation. The liquid formulation may further comprise an isotonic agent, such as mannitol, and/or a buffering agent. In some such embodiments, the buffering agent has a buffering capacity near physiological pH, such as between pH 6 and pH 10, or between pH 6 and pH 8, preferably between pH 6.0 and pH 7.5. Exemplary buffering agents include sodium bicarbonate, sodium citrate, glycine, or L-histidine. In some such embodiments, the sulfite compound is sodium metabisulfite and the buffering agent is L-histidine. In certain embodiments, the flexible polymeric infusion container comprises a polyolefin-based film, such as PolyCine APP 114.

The disclosure also provides methods for the treatment and/or management of pain using the parenteral liquid formulations described herein. The methods comprise parenterally administering to a subject in need of treatment and/or management of pain the parenteral liquid formulations described herein.

The disclosure also provides polymeric infusion containers filled with the parenteral liquid formulations described herein. In certain embodiments, the polymeric infusion containers and their contents have been terminally sterilized.

These and other objects of the invention are described in the following paragraphs. These objects should not be deemed to narrow the scope of the invention.

### BRIEF DESCRIPTION OF THE DRAWINGS

FIG. 1 is a process flow diagram of an exemplary compounding procedure.

FIG. 2A-2B are graphs showing pH over time for an exemplary acetaminophen/ibuprofen formulation subjected to terminal sterilization (TS) cycles of 15, 20, and 25 minutes, from pre-TS through 3 months storage under (A) long-term and (B) accelerated storage conditions.

FIG. 3A-3C are graphs showing APHA color over time for an exemplary acetaminophen/ibuprofen formulation subjected to terminal sterilization (TS) cycles of 15, 20, and 25 minutes, from post-TS through 9 or 6 months storage under (A) long-term, (B) intermediate, and (C) accelerated storage conditions.

FIG. 4A-4C are graphs showing acetaminophen assay results over time for an exemplary acetaminophen/ibuprofen formulation subjected to terminal sterilization (TS) cycles of 15, 20, and 25 minutes, from pre-TS through 3 months storage under (A) long-term, (B) intermediate, and (C) accelerated storage conditions.

FIG. 5A-5C are graphs showing ibuprofen assay results over time for an exemplary acetaminophen/ibuprofen formulation subjected to terminal sterilization (TS) cycles of 15, 20, and 25 minutes, from pre-TS through 3 months storage under (A) long-term, (B) intermediate, and (C) accelerated storage conditions.

FIG. 6 is a table showing growth of an unknown impurity having a relative retention time (RRT) of 1.66 over time for an exemplary acetaminophen/ibuprofen formulation subjected to terminal sterilization (TS) cycles of 15, 20, and 25 minutes, from pre-TS (Bulk) through 6 months storage under accelerated storage conditions.

### DETAILED DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. Definitions

As used in the specification and the appended claims, unless specified to the contrary, the following terms have the meaning indicated:

The term “about” refers generally to a range of numerical values (e.g., ±5 to 10% of the recited value) that one of ordinary skill in the art would consider equivalent to the recited value. Ranges can be expressed herein as from “about” one particular value, and/or to “about” another particular value. When such a range is expressed, the range is inclusive of the recited values.

The term “aqueous” refers to a composition, such as a formulation, that comprises water.

The term “initial” or “pre-TS” or “to” when used in connection with a given parameter such as APHA color, pH, acetaminophen assay, ibuprofen assay, or antioxidant concentration, refers to that parameter at the time the liquid formulation is filled into the polymeric infusion container, prior to subjecting the polymeric infusion container and liquid formulation therein composition to terminal sterilization.

The term “post-TS” when used in connection with a given parameter such as APHA color, pH, acetaminophen assay, ibuprofen assay, or antioxidant concentration, refers to that parameter at the time following subjecting the polymeric infusion container and liquid formulation therein composition to terminal sterilization, such as essentially immediately following terminal sterilization and up to two weeks following terminal sterilization. Unless otherwise indicated, the term “post-TS” refers to the parameter prior to storage under long-term, intermediate, or accelerated conditions.

The term “pharmaceutically acceptable” is used adjectivally to mean that the modified noun is appropriate for use as a pharmaceutical product for human use or as a part of a pharmaceutical product for human use.

The term “polyol” includes an organic compound containing two or more hydroxyl groups. Examples of polyols include, but are not limited to, mannitol and sorbitol. Sugar alcohols are polyols that are derived from sugars.

The term “subject” includes humans and other primates as well as other mammals. In certain embodiments, the subject is a human. The term subject includes, for example, a human adult suffering from or believed to be suffering from mild to moderate pain and/or in need of management of moderate to severe pain, particularly management of moderate to severe pain as an adjunct to opioid analgesics.

The term “sulfite compound” encompasses a compound that contains or is a source for a sulfite ion and includes, without limitation, sodium metabisulfite, sodium sulfite, and sodium bisulfite.

The terms “treat”, “treating” and “treatment” when used in the context of a method of using a particular formulation, refer to alleviating or abrogating a condition, disorder, or disease and/or the attendant symptoms thereof and, include, for example relief of mild to moderate pain and management of moderate to severe pain.

### B. Drug Substance

Liquid formulations disclosed herein comprise two active pharmaceutical ingredients: acetaminophen and ibuprofen or a pharmaceutically acceptable salt thereof. In one particular embodiment, the pharmaceutically acceptable salt of ibuprofen is ibuprofen sodium and, in particular, ibuprofen sodium dihydrate. In one particular embodiment, the liquid formulation comprises acetaminophen and ibuprofen sodium dihydrate.

Acetaminophen is chemically identified as N-acetyl-p-aminophenol. The molecular weight of acetaminophen 151.16 and its molecular formula is C₈H₉NO₂. Acetaminophen is a white, odorless, crystalline powder, possessing a slightly bitter taste. Acetaminophen is soluble in boiling water and 1N sodium hydroxide and is freely soluble in alcohol. Acetaminophen has the following chemical structure:

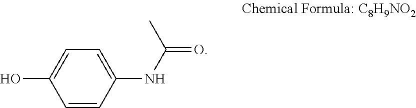

In certain embodiments, acetaminophen or a pharmaceutically acceptable salt thereof is present in the liquid formulation in an amount equivalent to 9.5 to 10.5 mg/mL acetaminophen or, alternatively, in an amount equivalent to 9.8 to 10.2 mg/mL acetaminophen or, alternatively, about 10.0 mg/mL acetaminophen. In some such embodiments, 9.5 to 10.5 mg/mL acetaminophen or, alternatively, 9.8 to 10.2 mg/mL acetaminophen or, alternatively, about 10.0 mg/mL acetaminophen is present in the liquid formulation.

Ibuprofen is chemically identified as 2-(p-isobutylphenyl)propionic acid. The molecular weight of ibuprofen 206.29 and its molecular formula is C₁₃H₁₈O₂. Ibuprofen is very poorly soluble in water. Ibuprofen has the following chemical structure:

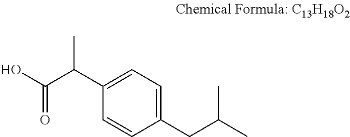

Ibuprofen sodium dihydrate is chemically identified as 2-(4-isobutyl phenyl) propionic acid sodium salt dihydrate. The molecular weight of ibuprofen sodium dihydrate 264.29 and its molecular formula is C₁₃H₂₁NaO₄. It is freely soluble in water. Ibuprofen sodium dihydrate has the following chemical structure:

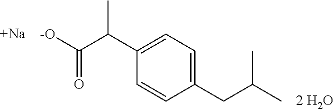

In certain embodiments, ibuprofen or a pharmaceutically acceptable salt thereof is present in the liquid formulation in an amount equivalent to 2.8 to 3.2 mg/mL ibuprofen or, alternatively, about 3.0 mg/mL ibuprofen. For example, a solution of 3.85 mg/mL of ibuprofen sodium dihydrate is equivalent to 3 mg/mL of ibuprofen.

In certain embodiments, the liquid formulation comprises 10 mg/mL acetaminophen and 3.85 mg/mL of ibuprofen sodium dihydrate (equivalent to 3.0 mg/mL of ibuprofen). In some such embodiments, the liquid formulation is contained within a flexible polymeric infusion container and contains 1000 mg acetaminophen and 385 mg of ibuprofen sodium dihydrate (equivalent to 300 mg of ibuprofen) per 100 mL.

In certain embodiments, the liquid formulation comprises a 3.3:1 (w/w) ratio of acetaminophen to ibuprofen.

### C. Liquid Formulations

This disclosure provides a liquid formulation that comprises acetaminophen and ibuprofen or a pharmaceutically acceptable salt thereof. The liquid formulation further comprises a sulfite compound as an antioxidant and a buffering agent. In some such embodiments, the buffering agent has a buffering capacity near physiological pH, such as between pH 6 and pH 10, or between pH 6 and pH 8, preferably between pH 6.0 and pH 7.5. Exemplary buffering agents include sodium bicarbonate, sodium citrate, glycine, or L-histidine. The liquid formulation may further comprise one or more excipients such as an isotonic agent and a pH adjusting agent.

This disclosure also provides an acetaminophen/ibuprofen liquid formulation that is suitable for terminal sterilization in a polymeric infusion container. The liquid formulation comprises acetaminophen, ibuprofen or a pharmaceutically acceptable salt thereof, a sulfite compound as an antioxidant, and a buffering agent. The liquid formulation is stable upon long-term storage following terminal sterilization.

The liquid formulations disclosed herein comprise acetaminophen and ibuprofen or a pharmaceutically acceptable salt thereof. In one particular embodiment, the pharmaceutically acceptable salt of ibuprofen is ibuprofen sodium and, in particular, ibuprofen sodium dihydrate. In one particular embodiment, the liquid formulation comprises acetaminophen and ibuprofen sodium dihydrate.

The liquid formulations disclosed herein comprise a sulfite compound as an antioxidant. The sulfite compound may be a sulfite salt or an organic sulfite. In certain embodiments, the sulfite compound is selected from the group consisting of sodium metabisulfite, sodium sulfite, and sodium bisulfite. In some such embodiments, the sulfite compound is sodium bisulfite. In some such embodiments, the sulfite compound is sodium sulfite. In some such embodiments, the sulfite compound is sodium metabisulfite.

Sodium metabisulfite has the molecular formula Na₂S₂O₅. Sodium sulfite has the molecular formula Na₂SO₃. Sodium bisulfite has the molecular formula NaHSO₃. The chemical structures of sodium metabisulfite, sodium sulfite, and sodium bisulfite are depicted below:

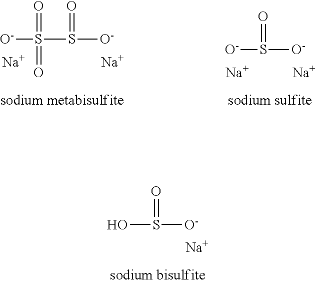

In certain embodiments, the sulfite compound is present in the liquid formulation in an amount from 0.0625 to 1.0 mg/mL, alternatively from 0.0625 to 0.5 mg/mL, at the time of preparation (to). In some such embodiments, the sulfite compound is present in an amount of 0.25 mg/mL at to.

The liquid formulations disclosed herein comprise a buffering agent. In some such embodiments, the buffering agent has a buffering capacity near physiological pH, such as between pH 6 and pH 10, or between pH 6 and pH 8, preferably between pH 6.0 and pH 7.5. In certain embodiments, the buffering agent comprises an amino acid. In some such embodiments, the amino acid buffering agent is selected from the group consisting of glycine and L-histidine. In certain embodiments, the buffering agent is selected from the group consisting of sodium bicarbonate, sodium citrate, glycine, and L-histidine. In some such embodiments, the buffering agent is sodium bicarbonate or sodium citrate. In other such embodiments, the buffering agent is glycine or L-histidine.

In certain embodiments, the buffering agent is present in the liquid formulation in an amount from 0.13 to 0.26 mg/mL at the time of preparation (to). In some such embodiments, the buffering agent is present in an amount of 0.13 mg/mL at to. In other such embodiments, the buffering agent is present in an amount of 0.26 mg/mL at to.

In certain embodiments, the antioxidant is sodium metabisulfite and the buffering agent is selected from the group consisting of sodium bicarbonate, sodium citrate, glycine, and L-histidine. In some such embodiments, the buffering agent is glycine or L-histidine. In some such embodiments, the antioxidant is sodium metabisulfite and the buffering agent is glycine. In other such embodiments, the antioxidant is sodium metabisulfite and the buffering agent is L-histidine.

The liquid formulations disclosed herein may comprise an isotonic agent. In certain embodiments, the isotonic agent comprises a sugar or a polyol. In some such embodiments, the isotonic agent comprises a sugar alcohol. In some such embodiments, the sugar alcohol has the general formula CH₂OH(CHOH)ₙCH₂OH, wherein n is from 2 to 5, such as 2, 3, 4, or 5, preferably 4. In certain embodiments, the isotonic agent is selected from the group consisting of mannitol, sorbitol, inositol, glucose and glycerol. In some such embodiments, the isotonic agent is mannitol or sorbitol. In some such embodiments, the isotonic agent is sorbitol. In some such embodiments, the isotonic agent is mannitol. In certain embodiments, the liquid formulation is isotonic.

The liquid formulations disclosed herein may comprise a pH adjusting agent. The pH adjusting agent that may comprise, without limitation, an acid such as sulfuric acid, phosphoric acid, or hydrochloric acid, a base such as sodium acetate, sodium bicarbonate, or sodium hydroxide, or a combination thereof. In certain embodiments, the pH adjusting agent is hydrochloric acid, sodium hydroxide, or a combination thereof. In certain embodiments, the pH of the liquid formulation is adjusted by addition of a pH adjusting agent. The concentration of the pH adjusting agent can be any concentration suitable for adjusting the pH, such as, for example, 1N acid or base. In certain embodiments, 1N hydrochloric acid and/or 1N sodium hydroxide are added to obtain the desired pH.

In certain embodiments, the liquid formulation has a pH in the range of about 6.0 to about 8.0, alternatively from about 6.0 to about 7.5, alternatively from about 6.3 to about 7.3, alternatively from about 6.8 to about 7.0, at the time of preparation (to). In an exemplary embodiment, the liquid formulation has a pH in the range of 6.0 to 7.5 at to. In another exemplary embodiment, the liquid formulation has a pH in the range of 6.3 to 7.3 at to. In still other exemplary embodiments, the liquid formulation has an initial pH in the range of 6.7 to 7.1 or 6.8 to 7.0, preferably about 6.9, at to.

In certain embodiments, the liquid formulations comprise an aqueous vehicle. In some such embodiments, the aqueous vehicle is Water For Injection.

In certain embodiments, the liquid formulation is an aqueous formulation. In certain embodiments, the liquid formulation comprises Water For Injection.

In certain embodiments, the liquid formulation comprises less than 0.25 mg/mL of a thiolic substance, such as cysteine, acetylcysteine, dithiothreitol or alpha-thioglycerol, thiomalic acid, thioglycerol, or methionine. In certain embodiments, the liquid formulation comprises less than 0.25 mg/mL of cysteine.

In certain embodiments, the liquid formulation is essentially free of a thiolic substance, such as cysteine, acetylcysteine, dithiothreitol or alpha-thioglycerol, thiomalic acid, thioglycerol, or methionine, as an antioxidant. In certain embodiments, the liquid formulation is essentially free of cysteine. In certain embodiments, the liquid formulation is essentially free of acetyl cysteine. In certain embodiments, the liquid formulation is essentially free of cysteine and acetyl cysteine.

In certain embodiments, the liquid formulation does not comprise a thiolic substance, such as cysteine, acetylcysteine, dithiothreitol or alpha-thioglycerol, thiomalic acid, thioglycerol, or methionine, as an antioxidant. In certain embodiments, the liquid formulation does not comprise cysteine. In certain embodiments, the liquid formulation does not comprise acetyl cysteine. In certain embodiments, the liquid formulation does not comprise cysteine and acetyl cysteine.

This disclosure provides a ready-to-administer parenteral liquid formulation comprising acetaminophen, ibuprofen or a pharmaceutically acceptable salt thereof, an isotonic agent, a buffering agent, an antioxidant, water for injection, and, optionally, one or more pH adjusting agents. An exemplary formulation is provided in Table 1 below.

TABLE 1

| Ingredient | Function | Concentration | |
|---|---|---|---|
| Acetaminophen, USP | Active Ingredient | 10 | mg/mL |
| Ibuprofen Sodium | Active Ingredient | 3.85 | mg/mL |
| Dihydrate, USP | | | |
| Mannitol, USP | Isotonic Agent | 32.85 | mg/mL |
| L-Histidine, USP | Buffering Agent | 0.195 | mg/mL |
| Sodium Metabisulfite, USP | Antioxidant | 0.25 | mg/mL |

| | | |
|---|---|---|
| 1N Sodium Hydroxide, NF | pH adjustment | q.s. |
| 1N Hydrochloric Acid, NF | pH adjustment | q.s. |
| Water For Injection | Aqueous Vehicle | q.s. |

### D. Polymeric Infusion Containers

The acetaminophen/ibuprofen liquid formulations disclosed herein are ideally terminally-sterilized, ready-to-administer (RTA) formulations for parenteral administration. The liquid formulations are stored in a pharmaceutically acceptable container, for example, an infusion container such as polymeric infusion container, preferably a flexible polymeric infusion container. Vehicles can include, for instance, fluids suitable for parenteral administration such as water for injection.

Exemplary materials for the containers and container components include, but are not limited to, polyolefins. Exemplary materials also include polysulfone, polycarbonate, polypropylene, polyethylene (such as low-density polyethylene (LDPE) or high density polyethylene (HDPE)), polystyrene, and co-polymers thereof. Preferably, the materials for the container and container components are suitable to withstand aseptic filling and not deform during terminal sterilization. In certain embodiments, the polymeric infusion container comprises a polyolefin/styrene block copolymer. In certain embodiments, the polymeric infusion container comprises a polyolefin-based film, and, in particular, the polyolefin based Advanced PolyPropylene (APP) manufactured by PolyCine.

The infusion container can be a flexible plastic container, optionally with ports and closure system for storing the liquid formulations. Infusion containers can include other conventional components, for example, connection ports, connector caps or connector disks.

The infusion containers may contain a port, such as a twist-off port. The infusion containers may be sealed with any appropriate closure, such as a twist-off stopper. An exemplary twist-off ports and/or stoppers may comprise polypropylene, such as those supplied by Technoflex® (e.g., a twist-off spike port made of polypropylene Inerta®).

In certain embodiments, the liquid formulation is prepared as a 100 mL fill packaged in a 100 mL flexible polymeric infusion container with a twist-off port.

In certain embodiments, the liquid formulation is contained within a flexible plastic infusion container made of PolyCine's three-layer film APP 114S, with a Technoflex® Twist-off Spike Port. In some such embodiments, this primary packaging will be placed inside secondary packaging comprising an aluminum overpouch (e.g., Polialuvel® from Wipf AG, where the overpouch has four layers in the following order: polyethylene terephthalate film, oriented polyamide film, aluminum, and cast polypropylene film; Climapac®). In some such embodiments, an oxygen scavenger, such as a StabilOx®D-100-H75 oxygen scavenger, is present inside the secondary packaging.

An exemplary flexible polymeric infusion container and closure system for the acetaminophen/ibuprofen liquid formulations disclosed herein is provided in Table 2 below.

TABLE 2

| Component | Description |
|---|---|
| IV | Container: 100 mL, PolyCine APP114 multilayer |
| Container | Polyolefin/Styrene block copolymer |
| Tube | Tube: PolyCine APP 107 Multilayer Polyolefin/Styrene |
| | Block Copolymer |
| Twist-off | Twist Off Spike Port, TP823, Polypropylene Inerta ® 016 |
| Port | |

### E. Compounding Procedure

In one aspect, this disclosure provides a method for manufacturing a product comprising an acetaminophen/ibuprofen liquid formulation packaged in a polymeric infusion container such as a flexible polymeric infusion container. In certain embodiments, the product comprises secondary packaging such as an aluminum overwrap.

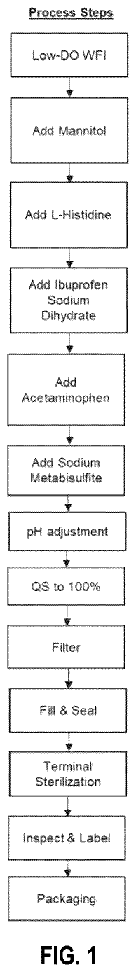

An exemplary compounding procedure is depicted in FIG. 1.

In certain embodiments, compounding takes place in a controlled environment in a stainless steel vessel equipped with a mixer.

In certain embodiments, dissolved oxygen levels are controlled via sparging with an inert gas, such as nitrogen, argon, or xenon, preferably nitrogen.

In certain embodiments, the acetaminophen/ibuprofen liquid formulation is filled into the polymeric infusion container under an inert gas, such as nitrogen, argon, or xenon, preferably nitrogen. Thus, in certain embodiments, the polymeric infusion container contains an inert gas layer (e.g., a nitrogen overlay).

In certain embodiments, the pH of the liquid formulation, upon filing into the flexible polymeric infusion container and in the absence of and/or prior to terminal sterilization, is 6.0 to 7.5, alternatively 6.3 to 7.3, alternatively 6.7 to 7.1 or 6.8 to 7.0, such as 6.9.

### F. Terminal Sterilization

General procedures for filling the liquid formulation into the polymeric infusion containers and their subsequent processing, for example, terminal sterilization, are known in the art.

Processing techniques of the formulations filled in an infusion container preferably use a sterilization process to destroy or eliminate any microorganisms that may be present in the acetaminophen/ibuprofen liquid formulations following preparation. For example, terminal sterilization can be used to destroy or eliminate any microorganisms that may be present in the sealed polymeric infusion container containing the acetaminophen/ibuprofen liquid formulation. A hot water cascade sterilizer or a steam autoclave are commonly used to accomplish terminal heat-sterilization of drug products in their final packaging.

In certain embodiments, the polymeric infusion container filled with the acetaminophen/ibuprofen liquid formulation is terminally sterilized using an autoclave. The liquid formulations in the polymeric infusion containers can be autoclaved at a temperature ranging from 115° C. to 130° C., preferably at a temperature ranging from 119° C. to 122° C., for a period of time ranging from about 3 to about 30 minutes, alternatively from about 5 to about 15 minutes, such as for 5, 6, 7, 8, 9, 10, 11, 12, 13, 14, or 15 minutes.

In certain embodiments, terminal sterilization can be characterized by specifying a F₀ value. The F₀ value is the equivalent time in minutes for the specified temperature that gives the same thermal lethality as at 121° C. For example, if a cycle has an F₀ value of 6, the sterilization effectiveness of that cycle is equal to 6 minutes at 121° C. regardless of the process temperature and time used in the cycle. In certain embodiments, the F₀ is calculation started at 118° C.

In some such embodiments, the F₀ is less than 20, such as from about 5 to about 15, such as 5, 6, 7, 8, 9, 10, 11, 12, 13, 14, or 15. In some such embodiments, the F₀ is not more than about 15.

As described herein, it was observed that the acetaminophen/ibuprofen liquid formulations contained in a polymeric infusion container can withstand terminal sterilization cycles up to F₀ of 15 minutes. Thus, in some such embodiments, the F₀ does not exceed 15.

### G. Appearance and Color

In at least one aspect, the liquid formulations disclosed herein have a clear, colorless appearance. In certain embodiments, the appearance of the liquid formulation is assessed visually. In certain embodiments, the visual appearance of the liquid formulation at the time of preparation (to) is a clear, colorless liquid. In certain embodiments, the visual appearance of the liquid formulation prior to terminal sterilization is a clear, colorless liquid. In certain embodiments, the visual appearance of the terminally sterilized liquid formulation is a clear, colorless liquid.

In at least one aspect, the APHA color for a liquid formulation is assessed. APHA color is a number calculated from spectrophotometric data that describes the change in color of a test sample from clear or white to yellow.

In certain embodiments, terminally sterilized liquid formulations disclosed herein have an APHA color of 250 or less, alternatively 200 or less, alternatively 150 or less, alternatively 100 or less. In some such embodiments, the terminally sterilized liquid formulations have an APHA color of 52 or less (e.g., immediately after terminal sterilization and for up to two weeks after terminal sterilization prior to long-term storage). In some such embodiments, the terminally sterilized liquid formulations have an APHA color of 128 or less (e.g., after storage for up to 3, 6, 9, 12, or 24 months under long-term storage conditions).

In certain embodiments, the APHA color of the liquid formulation post-TS and prior to long-term storage is 52 or less. In certain embodiments, the APHA color of the liquid formulation prior to long-term storage is 52 or less, wherein the liquid formulation has been terminally sterilized in a flexible polymeric infusion container. In certain embodiments, the APHA color of the liquid formulation prior to long-term storage is 52 or less, wherein the liquid formulation has been terminally sterilized in a flexible polymeric infusion container with an F₀ value of 15.

In certain embodiments, the APHA color of the liquid formulation after 3, 6, 9, 12, or 24 months under long-term storage conditions is 128 or less. In certain embodiments, the APHA color of the liquid formulation after 3, 6, 9, 12, or 24 months under long-term storage conditions is 128 or less, wherein the liquid formulation has been terminally sterilized in a flexible polymeric infusion container. In certain embodiments, the APHA color of the liquid formulation after 3, 6, 9, 12, or 24 months under long-term storage conditions is 128 or less, wherein the liquid formulation has been terminally sterilized in a flexible polymeric infusion container with an F₀ value of 15.

### H. Stability

Acetaminophen is heat and light sensitive and susceptible to oxidation. The formation of p-aminophenol following hydrolysis of acetaminophen which itself can degrade into quinoneimine is known.

In at least one aspect, the terminally sterilized liquid formulations disclosed herein are stable during, for example, storage, distribution, and the duration of the product's shelf-life (e.g., up to two years at room temperature/ambient conditions). A stable terminally sterilized liquid formulations may, for example, exhibit less degradation of the active ingredient(s) and/or low amounts of degradation products. Assay and degradation product determination of liquid formulations may be performed using High Performance Liquid Chromatography (“HPLC”) with UV detection.

In certain embodiments, the terminally sterilized liquid formulation packaged in a polymeric infusion container such as flexible polymeric infusion container meets release specifications and/or shelf-life specifications for acetaminophen and/or ibuprofen or a pharmaceutically acceptable salt thereof, such as ibuprofen sodium dihydrate. In some such embodiments, the amount of acetaminophen and/or ibuprofen sodium dihydrate is determined by HPLC.

Exemplary release specifications for acetaminophen are at least 93%, at least 94%, at least 95%, at least 96%, at least 97%, at least 98%, or at least 99% of the specified amount of acetaminophen (e.g., 10 mg/mL). In certain embodiments, release specifications for acetaminophen are from 95% to 105% of the specified amount of acetaminophen (e.g., 10 mg/mL).

Exemplary release specifications for ibuprofen sodium dihydrate are at least 93%, at least 94%, at least 95%, at least 96%, at least 97%, at least 98%, or at least 99% of the specified amount of ibuprofen sodium dihydrate (e.g., 3 mg/mL). In certain embodiments, release specifications for ibuprofen sodium dihydrate are from 95% to 105% of the specified amount of ibuprofen sodium dihydrate (e.g., 3 mg/mL).

Exemplary release specifications for APHA color are 150 or less, alternatively 100 or less. In certain embodiments, release specifications for APHA color are 52 or less.

In certain embodiments, the terminally sterilized acetaminophen/ibuprofen liquid formulation has an APHA color of 52 or less immediately after terminal sterilization and for up to one, two, three, or four weeks following terminal sterilization. In certain embodiments, the terminally sterilized acetaminophen/ibuprofen liquid formulation has an APHA color of 52 or less at the time of release testing.

Exemplary shelf-life specifications for acetaminophen are at least 90%, at least 91%, at least 92%, at least 93%, at least 94%, at least 95%, at least 96%, at least 97%, at least 98%, or at least 99% of the specified amount of acetaminophen (e.g., 10 mg/mL). In certain embodiments, release specifications for acetaminophen are from 95% to 105% of the specified amount of acetaminophen (e.g., 10 mg/mL).

Exemplary shelf-life specifications for ibuprofen sodium dihydrate are at least 90%, at least 91%, at least 92%, at least 93%, at least 94%, at least 95%, at least 96%, at least 97%, at least 98%, or at least 99% of the specified amount of ibuprofen sodium dihydrate (e.g., 3 mg/mL). In certain embodiments, release specifications for ibuprofen sodium dihydrate are from 95% to 105% of the specified amount of ibuprofen sodium dihydrate (e.g., 3 mg/mL).

Exemplary shelf-life specifications for APHA color are 250 or less, alternatively 200 or less, alternatively 150 or less. In certain embodiments, shelf-life specifications for APHA color are 128 or less.

Stability of a terminally sterilized acetaminophen/ibuprofen liquid formulation packaged in a polymeric infusion container such as a flexible polymeric infusion container may be assessed following storage for at least 1 month, at least 2 months, at least 3 months, at least 6 months, at least 9, at least 12 months, at least 15 months, at least 18 months, or at least 24 months. In particular, appearance, pH, color, osmolality, acetaminophen concentration, ibuprofen sodium dihydrate concentration, and/or antioxidant concentration may be assessed after storage for about 1, 2, 3, 6, 9, 12, 15, 18, and/or 24 months. Storage conditions may be long-term, intermediate, or accelerated storage conditions. In particular, storage conditions may be, for example, 25° C.±2° C. and 60% relative humidity (RH)+5% RH, 30° C.±2° C. and 65% RH±5% RH, and/or 40° C.±2° C. and 75% RH±5% RH.

In certain embodiments, the acetaminophen/ibuprofen liquid formulation is stable following terminal sterilization and storage for at least 1 month, at least 2 months, at least 3 months, or at least 6 months when stored at 25° C.±2° C. and 60% RH±5% RH.

In certain embodiments, the appearance of the terminally sterilized acetaminophen/ibuprofen liquid formulation is a clear, colorless liquid after storage for up to 1 month, up to 2 months, up to 3 months, or up to 6 months at 25° C.±2° C. and 60% RH±5% RH.

In certain embodiments, the appearance of the terminally sterilized acetaminophen/ibuprofen liquid formulation is a clear, colorless liquid after storage for up to 1 month, up to 2 months, or up to 3 months at 30° C.±2° C. and 65% RH±5% RH.

In certain embodiments, the appearance of the terminally sterilized acetaminophen/ibuprofen liquid formulation is a clear, colorless liquid after storage for up to 1 month or up to 2 months at 40° C.±2° C. and 75% RH±5% RH.

In certain embodiments, the terminally sterilized acetaminophen/ibuprofen liquid formulation has an APHA color of 128 or less after storage for up to 1 month, up to 2 months, up to 3 months, up to 6 months, or up to 9 months at 25° C.±2° C. and 60% RH±5% RH.

In certain embodiments, the terminally sterilized acetaminophen/ibuprofen liquid formulation has an APHA color of 128 or less after storage for up to 1 month, up to 2 months, up to 3 months, or up to 6 months at 30° C.±2° C. and 65% RH±5% RH.

In certain embodiments, the terminally sterilized acetaminophen/ibuprofen liquid formulation has an APHA color of 128 or less after storage for up to 1 month, up to 2 months, or up to 3 months at 40° C.±2° C. and 75% RH±5% RH.

In certain embodiments, the pH of the terminally sterilized acetaminophen/ibuprofen liquid formulation is from 6.0 to 7.5, alternatively, from 6.3 to 7.3, alternatively, from 6.5 to 7.1 after storage for up to 1 month, up to 2 months, up to 3 months, or up to 6 months at 25° C.±2° C. and 60% RH±5% RH or at 30° C.±2° C. and 65% RH±5% RH or at 40° C.±2° C. and 75% RH±5% RH.

In certain embodiments, the terminally sterilized acetaminophen/ibuprofen liquid formulation has at least 90%, alternatively at least 91%, alternatively at least 92%, alternatively at least 93%, alternatively at least 94%, alternatively at least 95%, such as from 95% to 105%, of the specified amount of acetaminophen (e.g., 10 mg/mL) after storage for up to 1 month, up to 2 months, up to 3 months, or up to 6 months at 25° C.±2° C. and 60% RH±5% RH or at 30° C.±2° C. and 65% RH±5% RH or at 40° C.±2° C. and 75% RH±5% RH.

In certain embodiments, the terminally sterilized acetaminophen/ibuprofen liquid formulation has at least 90%, alternatively at least 91%, alternatively at least 92%, alternatively at least 93%, alternatively at least 94%, alternatively at least 95%, such as from 95% to 105%, of the specified amount of ibuprofen sodium dihydrate (e.g., 3 mg/mL) after storage for up to 1 month, up to 2 months, up to 3 months, or up to 6 months at 25° C.±2° C. and 60% RH±5% RH or at 30° C.±2° C. and 65% RH±5% RH or at 40° C.±2° C. and 75% RH±5% RH.

### H. Methods of Use

In at least one aspect, this disclosure provides methods for treating and/or managing pain using the liquid formulations described herein. In certain embodiments, liquid formulations comprising acetaminophen and ibuprofen or a pharmaceutically acceptable salt thereof may be used to treat pain, including mild pain, moderate pain, and/or severe pain. In some such embodiments, liquid formulations described herein may be used to treat mild to moderate pain. In other such embodiments, liquid formulations described herein may be used to treat and/or manage moderate to severe pain. Liquid formulations comprising acetaminophen and ibuprofen or a pharmaceutically acceptable salt thereof may be used for the relief of mild to moderate pain. Liquid formulations comprising acetaminophen and ibuprofen or a pharmaceutically acceptable salt thereof may be used for the management of pain and, in particular, for the management of moderate to severe pain as an adjunct to opioid analgesics. Liquid formulations described herein may preferably be parenterally administered to a subject in need thereof. In particular, liquid formulations described herein may be intravenously administered to a subject in need thereof.

Thus, in certain embodiments, the methods comprise parenterally administering to a subject in need of treatment and/or management of pain an acetaminophen/ibuprofen liquid formulation, including a terminally-sterilized acetaminophen/ibuprofen liquid formulation, as described herein. In some such embodiments, the methods comprise intravenously administering to a subject in need of management of pain an acetaminophen/ibuprofen liquid formulation, including a terminally-sterilized acetaminophen/ibuprofen liquid formulation, as described herein.

In at least one aspect, this disclosure provides an acetaminophen/ibuprofen liquid formulation, including a terminally-sterilized acetaminophen/ibuprofen liquid formulation for use in a method for treating and/or managing pain. In certain embodiments, liquid formulations comprising acetaminophen and ibuprofen or a pharmaceutically acceptable salt thereof are for use in a method for treating treat pain, including mild pain, moderate pain, and/or severe pain. In some such embodiments, liquid formulations described herein are for use in a method for treating mild to moderate pain. In other such embodiments, liquid formulations described herein are for use in a method for treating and/or managing moderate to severe pain. Liquid formulations comprising acetaminophen and ibuprofen or a pharmaceutically acceptable salt thereof are for use in a method for relief of mild to moderate pain. Liquid formulations comprising acetaminophen and ibuprofen or a pharmaceutically acceptable salt thereof are for use in a method for management of pain and, in particular, for the management of moderate to severe pain as an adjunct to opioid analgesics. Liquid formulations described herein are preferably for parenteral administration to a subject in need thereof. In particular, liquid formulations described herein are for intravenous administration to a subject in need thereof.

Thus, in certain embodiments, the acetaminophen/ibuprofen liquid formulations, including terminally-sterilized acetaminophen/ibuprofen liquid formulations, are for parenteral administration to a subject in need of treatment and/or management of pain. In some such embodiments, the acetaminophen/ibuprofen liquid formulations, including terminally-sterilized acetaminophen/ibuprofen liquid formulations, are for intravenous administration to a subject in need of management of pain.

In certain embodiments, the subject is a human.

The liquid formulations, methods, and uses described herein will be better understood by reference to the following exemplary embodiments and examples, which are included as an illustration of and not a limitation upon the scope of the invention.

### I. Exemplary Embodiments

Exemplary Embodiment 1. A parenteral liquid formulation comprising: (a) 2.8 to 3.2 mg/ml ibuprofen; (b) 9.5 to 10.5 mg/ml acetaminophen; (c) an isotonic agent; (d) a buffering agent; and (e) an antioxidant, wherein the antioxidant comprises a sulfite compound; wherein the liquid formulation has a pH of 6.0 to 7.5 at the time of preparation (to).

Exemplary Embodiment 1.1. The liquid formulation of embodiment 1, wherein the acetaminophen is present in an amount from 9.8 to 10.2 mg/ml.

Exemplary Embodiment 1.2. The liquid formulation of embodiment 1, wherein the liquid formulation has a pH of 6.3 to 7.3 at to.

Exemplary Embodiment 2. The liquid formulation of embodiment 1, wherein the formulation has an APHA color of 128 or less following heat-sterilization in a flexible polymeric infusion container with an F₀ value of 15 and subsequent storage for up to 1, 2, 3, 6, 9, 12, or 24 months under long-term storage conditions.

Exemplary Embodiment 3. The liquid formulation of embodiment 2, wherein the flexible polymeric infusion container comprises a polyolefin and/or styrene block copolymer.

Exemplary Embodiment 4. The liquid formulation of any one of embodiments 1-3, wherein the sulfite compound is present in the liquid formulation in an amount from 0.0625 to 1.0 mg/ml at to.

Exemplary Embodiment 5. The liquid formulation of any one of embodiments 1-4, wherein the sulfite compound is selected from the group consisting of sodium metabisulfite, sodium sulfite, and sodium bisulfite.

Exemplary Embodiment 6. The liquid formulation of any one of embodiments 1-5, wherein the buffering agent comprises an amino acid.

Exemplary Embodiment 7. The liquid formulation of embodiment 6, wherein the amino acid is glycine or L-histidine.

Exemplary Embodiment 8. The liquid formulation of any one of embodiments 6 or 7, wherein the amino acid is present in the liquid formulation in an amount from 0.13 to 0.26 mg/ml at to.

Exemplary Embodiment 9. The liquid formulation of any one of embodiments 1-8, wherein the isotonic agent comprises mannitol.

Exemplary Embodiment 10. The liquid formulation of any one of embodiments 1-8, wherein the isotonic agent comprises mannitol, and the buffering agent comprises L-histidine.

Exemplary Embodiment 11. The liquid formulation of embodiment 10, where the antioxidant is sodium metabisulfite.

Exemplary Embodiment 12. The liquid formulation of any one of embodiments 1-11, wherein the liquid formulation does not comprise cysteine or acetyl cysteine.

Exemplary Embodiment 13. The liquid formulation of any one of embodiments 1-12, wherein the ratio of acetaminophen to ibuprofen is 3.3:1 (w/w).

Exemplary Embodiment 14. A flexible polymeric infusion container filled with at least 50 mL of the liquid formulation of any one of embodiments 1-13.

Exemplary Embodiment 15. A parenteral liquid formulation comprising: (a) 2.8 to 3.2 mg/ml ibuprofen; (b) 9.5 to 10.5 mg/ml acetaminophen; (c) an isotonic agent; (d) a buffering agent; and (e) an antioxidant, wherein the antioxidant comprises a sulfite compound; wherein the liquid formulation has been terminally sterilized in a flexible polymeric infusion container, wherein terminal sterilization is characterized by an F₀ value from 5 to 15; and wherein the liquid formulation has (a) an APHA color of 52 or less after terminal sterilization and/or (b) an APHA color of 128 or less after terminal sterilization and subsequent storage for up to 1, 2, 3, 6, 9, 12, or 24 months under long-term storage conditions.

Exemplary Embodiment 15.1. The liquid formulation of embodiment 15, wherein the acetaminophen is present in an amount from 9.8 to 10.2 mg/ml.

Exemplary Embodiment 16. The liquid formulation of embodiment 15, wherein the flexible polymeric infusion container comprises a polyolefin and/or styrene block copolymer.

Exemplary Embodiment 17. The liquid formulation of any one of embodiments 15 or 16, wherein the sulfite compound is present in the liquid formulation in an amount from 0.0625 to 1.0 mg/ml at the time of preparation (to) and in a reduced amount following terminal sterilization.

Exemplary Embodiment 18. The liquid formulation of any one of embodiments 15-17, wherein the buffering agent comprises an amino acid.

Exemplary Embodiment 19. The liquid formulation of embodiment 18, wherein the amino acid is glycine or L-histidine.

Exemplary Embodiment 20. The liquid formulation of any one of embodiments 18 or 19, wherein the amino acid is present in the liquid formulation in an amount from 0.13 to 0.26 mg/ml at to.

Exemplary Embodiment 21. The liquid formulation of any one of embodiments 15-20, wherein the liquid formulation does not comprise cysteine or acetyl cysteine.

Exemplary Embodiment 22. The liquid formulation of any one of embodiments 15-21, wherein the ratio of acetaminophen to ibuprofen is 3.3:1 (w/w).

Exemplary Embodiment 23. A flexible polymeric infusion container filled with at least 50 mL of the liquid formulation of any one of embodiments 15-22.

Exemplary Embodiment 24. A parenteral liquid formulation comprising: (a) 2.8 to 3.2 mg/ml ibuprofen; (b) 9.5 to 10.5 mg/ml acetaminophen; (c) 20 to 50 mg/ml of an isotonic agent; (d) 0.13 to 0.26 mg/ml of an amino acid buffering agent; and (e) an antioxidant, wherein the antioxidant comprises a sulfite compound, wherein the sulfite compound is present in the liquid formulation in an amount from 0.0625 to 1.0 mg/ml at the time of preparation (to); and wherein the liquid formulation has a pH of 6.0 to 7.5 at to.

Exemplary Embodiment 24.1. The liquid formulation of embodiment 24, wherein the acetaminophen is present in an amount from 9.8 to 10.2 mg/ml.

Exemplary Embodiment 24.2. The liquid formulation of embodiment 24, wherein the liquid formulation has a pH of 6.3 to 7.3 at to.

Exemplary Embodiment 25. The liquid formulation of embodiment 24, wherein the formulation has an APHA color of 128 or less following heat-sterilization in a flexible polymeric infusion container with an F₀ value of 15.

Exemplary Embodiment 26. The liquid formulation of embodiment 25, wherein the flexible polymeric infusion container comprises a polyolefin and/or styrene block copolymer.

Exemplary Embodiment 27. The liquid formulation of any one of embodiments 24-26, wherein the isotonic agent comprises mannitol.

Exemplary Embodiment 28. The liquid formulation of any one of embodiments 24-27, wherein the amino acid buffering agent comprises glycine or L-histidine.

Exemplary Embodiment 29. The liquid formulation of any one of embodiments 24-28, wherein the sulfite compound is selected from the group consisting of sodium metabisulfite, sodium sulfite, and sodium bisulfite.

Exemplary Embodiment 30. The liquid formulation of any one of embodiments 24-29, wherein the isotonic agent comprises mannitol and the buffering agent comprises L-histidine.

Exemplary Embodiment 31. The liquid formulation of embodiment 30, where the antioxidant is sodium metabisulfite.

Exemplary Embodiment 32. The liquid formulation of any one of embodiments 24-31, wherein the liquid formulation does not comprise cysteine or acetyl cysteine.

Exemplary Embodiment 33. The liquid formulation of any one of embodiments 24-32, wherein the ratio of acetaminophen to ibuprofen is 3.3:1 (w/w).

Exemplary Embodiment 34. A flexible polymeric infusion container filled with at least 50 mL of the liquid formulation of any one of embodiments 24-33.

Exemplary Embodiment 35. A pharmaceutical product comprising a stable, ready-to-use sterile parenteral liquid formulation of ibuprofen and acetaminophen packaged in a flexible polymeric infusion container, wherein the liquid formulation comprises: (a) 2.8 to 3.2 mg/ml ibuprofen; (b) 9.5 to 10.5 mg/ml acetaminophen; (c) an isotonic agent; (d) an amino acid buffering agent; and (e) an antioxidant, wherein the antioxidant comprises a sulfite compound.

Exemplary Embodiment 35.1. The liquid formulation of embodiment 1, wherein the acetaminophen is present in an amount from 9.8 to 10.2 mg/ml.

Exemplary Embodiment 36. The pharmaceutical product of embodiment 35, wherein the flexible polymeric infusion container comprises a polyolefin and/or styrene block copolymer.

Exemplary Embodiment 37. The pharmaceutical product of any one of embodiments 35 or 36, wherein the isotonic agent comprises mannitol.

Exemplary Embodiment 38. The pharmaceutical product of any one of embodiments 35-37, wherein the amino acid buffering agent comprises glycine or L-histidine.

Exemplary Embodiment 39. The pharmaceutical product of any one of embodiments 35-38, wherein the sulfite compound is selected from the group consisting of sodium metabisulfite, sodium sulfite, and sodium bisulfite.

Exemplary Embodiment 40. The pharmaceutical product of any one of embodiments 35-39, wherein the liquid formulation does not comprise cysteine or acetyl cysteine.

Exemplary Embodiment 41. The pharmaceutical product of any one of embodiments 35-40, wherein the ratio of acetaminophen to ibuprofen is 3.3:1 (w/w).

### J. Examples

In order to demonstrate the practice of the subject matter disclosed herein, the following examples have been prepared and tested. The examples should not, however, be viewed as limiting the scope of the invention.

### Materials and Methods

Primary and Secondary Packaging. The flexible polymeric infusion containers, tubes, and ports referenced in the below examples are described in Table 2 above. After terminal sterilization, the primary packaging was placed inside secondary packaging comprising an aluminum overwrap containing an oxygen scavenger.

Tests. The below examples employed the following tests:

Appearance was assessed visually.

pH was assessed according to USP <791>.

APHA Color (American Public Health Association color scale or Hazen scale) was assessed using a Hunterlab unit. Target Release Specification ≤52, Shelf-Life ≤128

Osmolality was assessed according to USP <785>.

Acetaminophen assay—Target Range: 95.0-105.0%, Preferred Range: 98.0-102.0% per USP monograph.

Ibuprofen sodium dihydrate assay—Target Range: 95.0-105.0%, Preferred Range: 97.0-103.0% per USP monograph.

L-cysteine levels were assessed by HPLC with a desired range of 60.0%-120.0% by HPLC.

Sodium metabisulfite levels were assessed by HPLC with a desired range of 0-50.0% Post terminal sterilization.

Total impurities were assessed by HPLC.

### Example 1: Terminal Sterilization of the Vial Formulation

This study characterized the impact of terminal sterilization on a commercially available acetaminophen/ibuprofen formulation comprising 10 mg/mL acetaminophen, 3.85 mg/mL ibuprofen sodium dihydrate, 32.85 mg/mL mannitol, 0.13 mg/mL disodium phosphate dihydrate, 0.25 mg/mL L-cysteine hydrochloride monohydrate, and water for injection as well as hydrochloric acid and sodium hydroxide (for pH adjustment).

COMBOGESIC® vials (reference listed product (RLD)) (100 cc) were obtained from AFT Pharmaceuticals Ltd. Each single-dose 100 mL vial contains 1,000 mg of acetaminophen and 300 mg of ibuprofen base (equivalent to 385 mg of ibuprofen sodium dihydrate), 25 mg of Cysteine hydrochloride monohydrate, 13 mg of Disodium phosphate dihydrate, 3285 mg of Mannitol, Hydrochloric acid (for pH adjustment), Sodium hydroxide (for pH adjustment), Water for injection.

The same formulation was prepared and filled into a 100 ml flexible polymeric infusion container (PolyCine APP 114 Multilayer Polyolefin/Styrene Block Copolymer).

The containers were subjected to terminal sterilization (TS) by exposure to steam in an autoclave for 15, 25, or 35 minutes (heated to 121° C. with an F₀ calculation starting at 118° C.). The resulting samples were analyzed for appearance, coloration, acetaminophen assay, ibuprofen sodium dihydrate assay, total impurities, and L-cysteine HCl.

The analytical results for these samples are listed in Table 3.

As shown in Table 3, terminal sterilization of the vial formulation in a flexible polymeric infusion container yielded a product where color was out of proposed specification after terminal sterilization; APHA color=100.1, 139.2, and 229.3 after terminal sterilization for 15, 25, and 35 minutes, respectively. The appearance of the product solutions were clear and free of visible particulates, the coloration of the product solution was yellow to brownish. Total impurities for these samples were also out of proposed specification after terminal sterilization.

Reducing the terminal sterilization time to 10.8 minutes with a formulation containing either 150% or 200% L-cysteine hydrochloride monohydrate (i.e., 0.375 or 0.5 mg/mL) resulted in a product where color was above the proposed specification after storage for 1 month under accelerated storage conditions.

TABLE 3
Analytical Profile of COMBOGESIC Formulation After Terminal
Sterilization (TS) in Vial or Polymeric Infusion Container

| | | | | | | Ibuprofen | | |
|---|---|---|---|---|---|---|---|---|
| | | | | | | Sodium | Total | |
| | Container | TS | | APHA | Acetaminophen | Dihydrate Assay | Impurities | L-Cysteine |
| Sample | Enclosure | (min) | Appearance | Coloration | Assay (%) | (%) | (%) | HCl (%) |
| Proposed | | | Clear Liquid | ≤52¹ | 95.0-105.0% | 95.0-105.0% | | 80-105% |
| Specification | | | | ≤128² | | | | |
| Combogesic IV | 100 cc | 15 | Clear | 0.0 | 101.60 | 100.40 | 0.7 | 71 |
| RLD | Vial | | | | | | | |
| Original | BULK | N/A | Clear | 2.2 | 103.60 | 104.20 | 0.06 | ND³ |
| Formula | 100 cc | 15 | Clear | 100.1 | 103.00 | 102.60 | 2.49 | <LOQ⁴ |
| | Infusion | 25 | Clear | 139.2 | 102.60 | 101.20 | 2.59 | <LOQ |
| | Container | 35 | Clear | 229.3 | 102.20 | 101.40 | 3.64 | <LOQ |

¹Proposed release specification
²Proposed Shelf-Life Specification
³Non-Detected
⁴Limit-of-quantification

### Example 2: Impact of Alternative Antioxidants

This study characterized the impact of different antioxidants on terminal sterilization of an acetaminophen/ibuprofen formulation comprising 10 mg/mL acetaminophen, 3.85 mg/mL ibuprofen sodium dihydrate, 32.85 mg/mL mannitol, and disodium phosphate dihydrate (buffer).

Formulations were prepared and filled into flexible polymeric infusion containers. The polymeric containers were subjected to terminal sterilization (TS) as described above for 20 and/or 25 minutes. The resulting samples were analyzed for, inter alia, appearance, color, and ibuprofen sodium dihydrate assay.

The antioxidants and antioxidant and buffer concentrations (mg/mL) used in these formulations and the analytical results for these samples are listed in Table 4.

As shown in Table 4, terminal sterilization of formulations containing sodium metabisulfite as the antioxidant in the flexible polymeric infusion containers yielded products where color was within the proposed specification after terminal sterilization.

TABLE 4

| | Conc. (mg/mL) | | Container | TS | |
|---|---|---|---|---|---|
| Antioxidant | Antioxidant | Buffer | Closure | (min) | Comment |
| Citric Acid | 0.26 | 0.13 | Polymeric | 25 | APHA color = 189.8 above proposed specification post-TS; product |
| | | | | | coloration brown/yellow post-TS |
| | 1.0 | 0.13 | Polymeric | 25 | APHA color = 260.1 above proposed specification post-TS; product |
| | | | | | coloration brown/yellow post-TS |
| Ascorbic Acid | 0.26 | 0.13 | Polymeric | 25 | APHA color = 71.6 above proposed specification post-TS; product |
| | | | | | coloration brown/yellow post-TS ** |
| | 1.0 | 0.13 | Polymeric | 25 | APHA color = 134.5 above proposed specification post-TS; product |
| | | | | | coloration brown/yellow post-TS |
| L-cysteine + sodium | 0.25 + 0.25 | 0.13 | Polymeric | 20 | APHA color = 145.3; Product coloration brown/yellow after 1 M accelerated |
| metabisulfite | | | | | stability |
| sodium metabisulfite + | 0.25 + 0.25 | 0.13 | Polymeric | 20 | APHA color = 2.8; Product coloration clear colorless after 1 M accelerated |
| L-methionine | | | | | stability but loss of −3.5% ibuprofen assay was observed |
| sodium metabisulfite | 0.26 | 0.13 | Polymeric | 25 | APHA color = 11.2 within proposed release specification post-TS; product |
| | | | | | coloration appears as clear/colorless |
| | 1.0 | 0.13 | Polymeric | 25 | APHA color = 6.3 within proposed release specification post-TS; product |
| | | | | | coloration appears as clear/colorless |
| | 0.25 | 0.13 | Polymeric | 20 | Loss of −4.5% ibuprofen assay was observed post-TS; APHA color = 32.8 |
| | | | | | within proposed release specification; product coloration appears as clear |
| | | | | | and slightly yellow |
| | 0.125 | 0.13 | Polymeric | 20 | Loss of −3.8% ibuprofen assay was observed post-TS; APHA color = 49.8 |
| | | | | | within proposed release specification; product coloration appears pink post-TS |
| | 0.0625 | 0.13 | Polymeric | 20 | Loss of −4.0% ibuprofen assay was observed post-TS; APHA color = 59.2 |
| | | | | | above proposed release specification Post-TS; product coloration |
| | | | | | brown/yellow post-TS |
| | 0.25 | 0.1365 | Polymeric | 20 | Loss of −6.82% ibuprofen Post-TS; APHA color = 46.0 within proposed |
| | | | | | release specification Post-TS; product coloration appears as clear and |
| | | | | | slightly yellow |
| | 0.125 | 0.1365 | Polymeric | 20 | Loss of −5.8% ibuprofen Post-TS; APHA color = 43.8 within proposed |
| | | | | | release specification Post-TS; product coloration brown/yellow post-TS |
| | 0.0625 | 0.1365 | Polymeric | 20 | Loss of −4.6% ibuprofen Post-TS, APHA color = 68.6 above proposed |
| | | | | | release specification; product coloration brown/yellow post-TS |

** It was determined that due to an ascorbic acid degradant being colored this would confuse this analysis as it could increase over time.

### Example 3: Impact of Buffer Component with Sulfite Antioxidant

This study characterized the impact of different buffer components on terminal sterilization of an acetaminophen/ibuprofen formulation comprising 10 mg/mL acetaminophen, 3.85 mg/mL ibuprofen sodium dihydrate, 32.85 mg/mL mannitol, and 0.25 mg/mL sodium metabisulfite (antioxidant).

Formulations were prepared and filled into flexible polymeric infusion containers. The containers were subjected to terminal sterilization (TS) as described above. Formulations containing 0.26 mg/mL L-histidine were subjected to TS for 25, 30, or 35 minutes. The resulting samples were analyzed for, inter alia, appearance, color, acetaminophen assay, ibuprofen sodium dihydrate assay, and total impurities.

The buffer components and concentrations (mg/mL) used in these formulations and the analytical results for these samples are listed in Table 5.

As shown in Table 5, each buffer component showed some degree of maintaining stability of the product during terminal sterilization comparatively to the disodium phosphate dihydrate.

TABLE 5

| Buffer | Buffer Conc. | TS | pH | | Ibuprofen Assay (%) | | |
|---|---|---|---|---|---|---|---|
| Component | (mg/mL) | (min) | Pre-TS | Post-TS | Pre-TS | Post-TS | Comment |
| Disodium | 0.13 | 20 | 6.4 | 6.0 | 103.5% | 92.5% | APHA Color = 14.4; Product Clear Colorless |
| Phosphate | | 15 | 6.7 | 6.2 | 101.0% | 97.4% | APHA Color = 0.0; Product Clear Colorless |
| Dihydrate | | 25 | 6.7 | 6.1 | 101.0% | 97.4% | APHA Color = 1.3; Product Clear Colorless |
| Sodium | 0.13 | 13 | 6.7 | 6.0 | 99.9% | 97.6% | APHA Color = 7.1; Product Clear Colorless |
| Bicarbonate | 0.13 | 20 | 6.7 | 6.0 | | 98.2% | APHA Color = 11.4; Product Clear Colorless |
| Sodium Citrate | 0.13 | 13 | 6.7 | 6.1 | 99.2% | 101.1% | APHA Color = 4.3; Product Clear Colorless |
| | 0.13 | 20 | 6.7 | 6.1 | | 100.0% | APHA Color = 5.8; Product Clear Colorless |
| Glycine | 0.13 | 15 | 6.9 | 6.4 | 100.8% | 100.1% | APHA Color = 1.6; Product Clear Colorless |
| | | 20 | | 6.4 | | 100.1% | APHA Color = 1.6; Product Clear Colorless |
| | | 25 | | 6.5 | | 99.2% | APHA Color = 0.0; Product Clear Colorless |
| | 0.26 | 15 | 6.9 | 6.3 | 101.2% | 99.1% | APHA Color = 1.5; Product Clear Colorless |
| | | 20 | | 6.3 | | 99.0% | APHA Color = 1.8; Product Clear Colorless |
| | | 25 | | 6.3 | | 99.2% | APHA Color = 2.0; Product Clear Colorless |
| L-Histidine | 0.13 | 13 | 6.7 | 6.4 | 101.9% | 100.5% | APHA Color = 0.0; Product Clear Colorless |
| | | 15 | 6.9 | 6.5 | 101.0% | 99.8% | APHA Color = 1.5; Product Clear Colorless |
| | | 20 | | 6.5 | | 100.1% | APHA Color = 1.3; Product Clear Colorless |
| | | 25 | | 6.5 | | 99.0% | APHA Color = 1.5; Product Clear Colorless |
| | 0.157 | 15 | 6.9 | 6.6 | 101.7% | 101.2% | APHA Color = 1.6; Product Clear Colorless |
| | | 20 | | 6.6 | | 101.0% | APHA Color = 1.3; Product Clear Colorless |
| | | 25 | | 6.6 | | 100.2% | APHA Color = 1.3; Product Clear Colorless |
| | 0.195 | 15 | 6.9 | 6.8 | 100.9% | 101.0% | APHA Color = 2.3; Product Clear Colorless |
| | | 20 | | 6.8 | | 100.4% | APHA Color = 2.1; Product Clear Colorless |
| | | 25 | | 6.8 | | 100.0% | APHA Color = 2.3; Product Clear Colorless |

### Example 4: Stability of Formulations Containing Sulfite Antioxidant

For terminal sterilization feasibility, five formulations were tested.

Each formulation contained 10 mg/mL acetaminophen, 3.85 mg/mL ibuprofen sodium dihydrate, 32.85 mg/mL mannitol, and 0.25 mg/mL sodium metabisulfite. Additionally, the formulations contained glycine or L-histidine as a buffering agent. The buffering agent and concentrations are shown in Table 6.

TABLE 6

| Formulation # | Buffering Agent | Buffer Conc. (mg/mL) |
|---|---|---|
| Formulation 1 | L-histidine | 0.13 mg/mL |
| Formulation 2 | L-histidine | 0.157 mg/mL |
| Formulation 3 | L-histidine | 0.195 mg/mL |
| Formulation 4 | glycine | 0.13 mg/mL |
| Formulation 5 | glycine | 0.26 mg/mL |

In this study, each formulation was compounded at an initial pH of 6.9.

Samples were assessed for appearance, pH, color, osmolality, acetaminophen assay, ibuprofen sodium dihydrate assay, and sodium metabisulfite after compounding, as bulk solution (unfiltered, pre-exposure to bioburden reduction filter), filter flush (tested to determine compatibility with filter contact materials), and bulk solution (filtered).

Proposed acceptance criteria for certain of these parameters are listed in Table 7.

TABLE 7

| Test | Proposed Acceptance Criteria |
|---|---|
| Appearance | Clear colorless to pale yellow liquid |
| | essentially free of visible particles |
| pH | 6.0-7.5 |
| APHA Color | At T0: ≤52, Shelf Life: ≤128 |
| Acetaminophen Assay | 95.0-105.0% |
| Ibuprofen Sodium Dihydrate Assay | 95.0-105.0% |

The analytical results for these samples are listed in Table 8.

TABLE 8

| | Formulation 1 | | | Formulation 2 | | | Formulation 3 | | | Formulation 4 | | | Formulation 5 | | |
|---|---|---|---|---|---|---|---|---|---|---|---|---|---|---|---|
| | Bulk | Filter | Bulk | Bulk | Filter | Bulk | Bulk | Filter | Bulk | Bulk | Filter | Bulk | Bulk | Filter | Bulk |
| Test | (UF) | Flush | (F) | (UF) | Flush | (F) | (UF) | Flush | (F) | (UF) | Flush | (F) | (UF) | Flush | (F) |
| Appearance | Clear, colorless liquid | | | Clear, colorless liquid | | | Clear, colorless liquid | | | Clear, colorless liquid | | | Clear, colorless liquid | | |
| pH | 6.1 | 6.2 | 6.2 | 6.2 | 6.2 | 6.6 | 6.4 | 6.3 | 6.7 | 6.2 | 6.1 | 6.8 | 6.0 | 6.1 | 6.3 |
| APHA Color | 6.5 | 0.7 | 1.5 | 6.7 | 2.4 | 1.1 | 5.6 | 2.2 | 1.1 | 6.0 | 1.1 | 1.5 | 8.8 | 1.1 | 0.9 |
| Osmolality | 289 | 289 | 288 | 289 | 292 | 292 | 293 | 291 | 294 | 292 | 287 | 290 | 294 | 293 | 294 |
| Acetaminophen Assay | 100.3 | 100.0 | 99.8 | 100.3 | 99.9 | 100.6 | 99.8 | 99.5 | 100.1 | 100.4 | 96.9 | 98.5 | 100.6 | 99.8 | 99.3 |
| (%) | | | | | | | | | | | | | | | |
| Ibuprofen Assay (%) | 101.4 | 101.2 | 101.0 | 101.4 | 101.2 | 101.7 | 101.2 | 100.6 | 100.9 | 102.0 | 98.4 | 100.8 | 101.9 | 101.3 | 101.2 |

Samples were filled into flexible polymeric infusion containers and subjected to a 15-, 20- and 25-minute terminal sterilization cycle. (F₀ calc start at 118° C., Target F₀=121° C.).

Samples were then placed on long-term (25±2° C. and 60±5% Relative Humidity (RH)), intermediate (30±2° C. and 65±5% RH), and accelerated (40±2° C. and 75±5% RH) storage conditions.

Samples were assessed by acetaminophen assay and ibuprofen sodium dihydrate assay at 0 (post-TS), 1, 2, 3, 6, and 9 months. Appearance, pH, color, osmolality, and sodium metabisulfite were also assessed.

Composition of Combogesic IV with
L-Histidine Buffer Formulas 1-3

| Component | Concentration | Function |
|---|---|---|
| Acetaminophen, USP | 10.0 mg/mL | Active |
| Ibuprofen Sodium | 3.85 mg/mL (3 mg/mL | Active |
| Dihydrate, USP | as Ibuprofen) | |
| Mannitol, USP | 32.85 mg/mL | Isotonic Agent |
| L-Histidine, USP | 1. 0.13 mg/mL | Buffer |
| | 2. 0.157 mg/mL | |
| | 3. 0.195 mg/mL | |
| Sodium Metabisulfite NF | 0.25 mg/mL | Antioxidant |
| Sodium Hydroxide, NF | q.s. (compounding pH | pH Adjustor |
| (used as 1N solution) | target 6.9, shelf-life | |
| Hydrochloric Acid, NF | range pH 6.0-7.3) | pH Adjustor |
| (used as 1N solution) | | |
| Water for Injection, USP | q.s. to 1 mL | Vehicle |
| Nitrogen, NF | N/A | Inert Atmosphere |

TABLE 1
Composition of Combogesic IV with
Glycine Buffer Formulas 4 + 5

| Component | Concentration | Function |
|---|---|---|
| Acetaminophen, USP | 10.0 mg/mL | Active |
| Ibuprofen Sodium | 3.85 mg/mL (3 mg/mL | Active |
| Dihydrate, USP | as Ibuprofen) | |
| Mannitol, USP | 32.85 mg/mL | Isotonic Agent |
| Glycine, USP | 4. 0.13 mg/mL | Buffer |
| | 5. 0.26 mg/mL | |
| Sodium Metabisulfite, NF | 0.25 mg/mL | Antioxidant |
| Sodium Hydroxide, NF | q.s. (compounding pH | pH Adjustor |
| (used as 1N solution) | target 6.9, shelf-life | |
| Hydrochloric Acid, NF | range pH 6.0-7.3) | pH Adjustor |
| (used as 1N solution) | | |
| Water for Injection, USP | q.s. to 1 mL | Vehicle |
| Nitrogen, NF | N/A | Inert Atmosphere |

For Formulation 1, appearance was a clear, colorless liquid under long-term storage conditions up to 6 months, regardless of TS duration. Appearance was a clear, colorless liquid under intermediate storage conditions up to 3 months, regardless of TS duration, with the appearance becoming clear, slightly yellow/brown liquid by 6 months. Appearance was a clear, colorless liquid under accelerated storage conditions up to 2 months, regardless of TS duration, with the appearance becoming clear, yellow/brown liquid by 3 months.

pH appeared controlled post-TS (loss of −0.4 units) from 6.9 to 6.5. Under accelerated storage conditions the pH showed identical drop but then begins to increase from 6.5 to 6.6.

Color was controlled under long-term storage conditions over 2 months but at accelerated storage conditions color slightly increased starting at 1 month to 2 months.

Acetaminophen assay was stable up to 6 months under long-term, intermediate, and accelerated stability conditions. Ibuprofen appears stable under long-term and intermediate storage conditions up to 6 months however a decrease (−4.3%) was observed at accelerated storage conditions over 6 months.

A single impurity appeared to be growing under accelerated storage conditions.

Tables 9-11 show stability under long-term, intermediate, and accelerated storage conditions over time for Formulation 1 after terminal sterilization for 15, 20, and 25 minutes.

TABLE 9
(Formulation 1; TS 15 min).

| | Formulation 1-100 mL Filled Polymeric Infusion Container in Overwrap with Oxygen Scavenger - TS 15 min | | | | | | | | | | | | | | |
|---|---|---|---|---|---|---|---|---|---|---|---|---|---|---|---|
| | | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | Post-TS | 1 | 2 | 3 | 6 | 9 | 1 | 2 | 3 | 6 | 9 | 1 | 2 | 3 | 6 |
| pH | 6.5 | 6.41 | 6.38 | 6.38 | 6.36 | 6.45 | 6.42 | 6.43 | 6.42 | 6.53 | 6.53 | 6.57 | 6.61 | 6.50 | 6.59 |
| APHA Color | 1.5 | 1.5 | 3.5 | 1.3 | 9.5 | 45.4 | 0.9 | −1.0 | 5.0 | 69.9 | 126.5 | 3.7 | 7.5 | 98.8 | 171.8 |
| Acet- | 100.2 | 100.6 | 100.9 | 100.4 | 99.6 | 100.2 | 100.2 | 100.7 | 99.3 | 99.3 | 100.4 | 100.3 | 99.7 | 99.1 | 99.1 |
| aminophen | | | | | | | | | | | | | | | |
| (%) | | | | | | | | | | | | | | | |
| Ibuprofen (%) | 99.8 | 100.2 | 100.2 | 98.0 | 98.5 | 97.9 | 99.5 | 99.8 | 96.5 | 97.4 | 97.0 | 98.4 | 97.2 | 95.4 | 96.1 |
| Sodium | 14.1 | 7.1 | 10.5 | — | 1.7 | — | 5.6 | 9.8 | — | 1.7 | — | 2.3 | 7.5 | — | 1.7 |
| Metabisulfite | | | | | | | | | | | | | | | |
| (%) | | | | | | | | | | | | | | | |

TABLE 10
(Formulation 1; TS 20 min).

| | Formulation 1-100 mL Filled Polymeric Infusion Container in Overwrap with Oxygen Scavenger - TS 20 min | | | | | | | | | | | | | | |
|---|---|---|---|---|---|---|---|---|---|---|---|---|---|---|---|
| | | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | Post-TS | 1 | 2 | 3 | 6 | 9 | 1 | 2 | 3 | 6 | 9 | 1 | 2 | 3 | 6 |
| pH | 6.5 | 6.43 | 6.40 | 6.40 | 6.41 | 6.45 | 6.43 | 6.41 | 6.45 | 6.48 | 6.51 | 6.55 | 6.59 | 6.53 | 6.58 |
| APHA Color | 1.3 | 1.1 | 3.0 | 1.6 | 6.7 | 45.1 | 1.6 | 6.0 | 3.3 | 71.6 | 137.7 | 6.7 | 65.6 | 89.2 | 178.9 |
| Acetaminophen | 100.8 | 100.4 | 100.5 | 100.1 | 99.3 | 99.6 | 100.1 | 99.6 | 99.7 | 98.9 | 101.0 | 100.5 | 99.9 | 99.6 | 99.5 |
| (%) | | | | | | | | | | | | | | | |
| Ibuprofen (%) | 100.1 | 99.8 | 100.0 | 97.9 | 98.1 | 97.3 | 99.4 | 98.4 | 96.8 | 97.1 | 97.8 | 98.6 | 97.5 | 95.6 | 96.7 |
| Sodium | 14.1 | 7.6 | 10.8 | — | 1.8 | — | 6.5 | 9.2 | — | 1.7 | — | 2.0 | 7.2 | — | 1.7 |
| Metabisulfite | | | | | | | | | | | | | | | |
| (%) | | | | | | | | | | | | | | | |

TABLE 11
(Formulation 1; TS 25 min).

| | Formulation 1-100 mL Filled Polymeric Infusion Container in Overwrap with Oxygen Scavenger - TS 25 min | | | | | | | | | | | | | | |
|---|---|---|---|---|---|---|---|---|---|---|---|---|---|---|---|
| | | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | Post-TS | 1 | 2 | 3 | 6 | 9 | 1 | 2 | 3 | 6 | 9 | 1 | 2 | 3 | 6 |
| pH | 6.5 | 6.47 | 6.43 | 6.41 | 6.43 | 6.43 | 6.44 | 6.43 | 6.44 | 6.54 | 6.53 | 6.58 | 6.71 | 6.55 | 6.56 |
| APHA Color | 1.5 | 1.6 | 2.4 | −1.0 | 8.6 | 51.3 | 2.8 | 3.3 | 3.3 | 63.7 | 142.6 | 4.1 | 13.5 | 102.6 | 180.2 |
| Acetaminophen | 99.9 | 100.4 | 100.1 | 99.8 | 99.1 | 101.2 | 99.5 | 99.5 | 99.6 | 98.8 | 100.5 | 100.7 | 99.8 | 99.3 | 99.3 |
| (%) | | | | | | | | | | | | | | | |
| Ibuprofen (%) | 99.0 | 99.9 | 99.3 | 97.5 | 97.9 | 98.8 | 98.8 | 98.3 | 96.6 | 96.7 | 97.2 | 98.4 | 96.7 | 95.4 | 96.7 |
| Sodium | 12.8 | 7.8 | 10.7 | — | 1.8 | — | 6.1 | 9.1 | — | 1.7 | — | 2.7 | 7.3 | — | 1.7 |
| Metabisulfite | | | | | | | | | | | | | | | |
| (%) | | | | | | | | | | | | | | | |

For Formulation 2, appearance was a clear, colorless liquid under long-term storage conditions up to 6 months, regardless of TS duration. Appearance was a clear, colorless liquid under intermediate storage conditions up to 3 months, regardless of TS duration, with the appearance becoming clear, slightly yellow/brown liquid by 6 months. Appearance was a clear, colorless liquid under accelerated storage conditions up to 2 months, regardless of TS duration, with the appearance becoming clear, yellow/brown liquid by 3 months.

pH appeared controlled post-TS (loss of −0.3 units) from 6.9 to 6.6.

Color was controlled under long-term storage conditions over 2 months but under accelerated storage conditions color slightly increased starting at 1 month to 2 months.

Acetaminophen assay was stable up to 6 months under long-term, intermediate, and accelerated stability conditions. Ibuprofen appeared stable under long-term and intermediate storage conditions up to 6 months however a decrease (−2.8%) was observed at accelerated storage conditions over 6 months.

Tables 12-14 show stability under long-term, intermediate, and accelerated storage conditions over time for Formulation 2 after terminal sterilization for 15, 20, and 25 minutes.

TABLE 12
(Formulation 2; TS 15 min).

| | Formulation 2-100 mL Filled Polymeric Infusion Container in Overwrap with Oxygen Scavenger - TS 15 min | | | | | | | | | | | | | | |
|---|---|---|---|---|---|---|---|---|---|---|---|---|---|---|---|
| | Post- | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | TS | 1 | 2 | 3 | 6 | 9 | 1 | 2 | 3 | 6 | 9 | 1 | 2 | 3 | 6 |
| pH | 6.6 | 6.45 | 6.41 | 6.41 | 6.44 | 6.43 | 6.44 | 6.43 | 6.48 | 6.53 | 6.52 | 6.53 | 6.59 | 6.52 | 6.57 |
| APHA Color | 1.6 | 1.8 | 5.8 | −0.8 | 6.3 | 51.8 | 3.0 | 4.7 | 5.4 | 72.1 | 141.9 | 3.7 | 69.5 | 103.9 | 178.9 |
| Acet- | 101.3 | 101.2 | 101.1 | 99.4 | 98.6 | 101.1 | 100.2 | 100.6 | 100.1 | 100.2 | 100.9 | 100.1 | 101.4 | 101.2 | 100.0 |
| aminophen | | | | | | | | | | | | | | | |
| (%) | | | | | | | | | | | | | | | |
| Ibuprofen | 101.2 | 101.1 | 100.8 | 97.5 | 97.9 | 99.2 | 99.9 | 100.1 | 97.3 | 98.7 | 98.2 | 98.8 | 99.2 | 97.8 | 97.6 |
| (%) | | | | | | | | | | | | | | | |
| Sodium | 17.9 | 7.7 | 10.4 | — | 1.7 | — | 6.0 | 9.2 | — | 1.7 | — | 2.5 | 7.1 | — | 1.7 |
| Metabisulfite | | | | | | | | | | | | | | | |
| (%) | | | | | | | | | | | | | | | |

TABLE 13
(Formulation 2; TS 20 min).

| | Formulation 2-100 mL Filled Polymeric Infusion Container in Overwrap with Oxygen Scavenger - TS 20 min | | | | | | | | | | | | | | |
|---|---|---|---|---|---|---|---|---|---|---|---|---|---|---|---|
| | Post- | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | TS | 1 | 2 | 3 | 6 | 9 | 1 | 2 | 3 | 6 | 9 | 1 | 2 | 3 | 6 |
| pH | 6.6 | 6.59 | 6.46 | 6.43 | 6.43 | 6.43 | 6.53 | 6.47 | 6.46 | 6.52 | 6.54 | 6.54 | 6.63 | 6.53 | 6.58 |
| APHA | 1.3 | 1.1 | 2.4 | −0.6 | 3.9 | 42.1 | 2.4 | 4.3 | 1.6 | 75.5 | 153.3 | 2.2 | 37.7 | 117.2 | 205.2 |
| Color | | | | | | | | | | | | | | | |
| Acet- | 100.8 | 101.2 | 101.1 | 100.4 | 99.8 | 101.4 | 101.0 | 101.5 | 100.1 | 99.5 | 101.0 | 101.1 | 101.0 | 101.4 | 99.6 |
| aminophen | | | | | | | | | | | | | | | |
| (%) | | | | | | | | | | | | | | | |
| Ibuprofen | 101.0 | 101.5 | 101.5 | 99.1 | 99.5 | 99.7 | 101.4 | 101.1 | 97.4 | 98.0 | 98.3 | 100.1 | 98.9 | 98.1 | 97.3 |
| (%) | | | | | | | | | | | | | | | |
| Sodium | 27.5 | 18.1 | 16.6 | — | 2.7 | — | 16.1 | 12.0 | — | 1.7 | — | 3.2 | 7.2 | — | 1.7 |
| Metabisulfite | | | | | | | | | | | | | | | |
| (%) | | | | | | | | | | | | | | | |

TABLE 14
(Formulation 2; TS 25 min).

| | Formulation 2-100 mL Filled Polymeric Infusion Container in Overwrap with Oxygen Scavenger - TS 25 min | | | | | | | | | | | | | | |
|---|---|---|---|---|---|---|---|---|---|---|---|---|---|---|---|
| | Post- | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | TS | 1 | 2 | 3 | 6 | 9 | 1 | 2 | 3 | 6 | 9 | 1 | 2 | 3 | 6 |
| pH | 6.6 | 6.59 | 6.57 | 6.44 | 6.42 | 6.43 | 6.58 | 6.47 | 6.44 | 6.51 | 6.55 | 6.58 | 6.61 | 6.53 | 6.57 |
| APHA Color | 1.3 | 1.1 | 1.8 | 0.1 | 3.2 | 37.4 | 1.6 | 3.5 | 1.6 | 72.9 | 147.9 | 2.4 | 53.2 | 115.4 | 204.1 |
| Acet- | 100.4 | 100.2 | 100.5 | 100.9 | 98.8 | 100.5 | 99.6 | 101.2 | 100.2 | 99.5 | 101.8 | 100.8 | 101.1 | 100.0 | 100.3 |
| aminophen | | | | | | | | | | | | | | | |
| (%) | | | | | | | | | | | | | | | |
| Ibuprofen | 100.2 | 100.6 | 100.9 | 99.6 | 98.7 | 98.9 | 100.0 | 101.2 | 98.3 | 98.1 | 98.8 | 99.6 | 99.2 | 96.8 | 98.2 |
| (%) | | | | | | | | | | | | | | | |
| Sodium | 24.4 | 18.9 | 22.9 | — | 3.0 | — | 17.1 | 13.7 | — | 1.7 | — | 4.7 | 7.2 | — | 1.7 |
| Metabisulfite | | | | | | | | | | | | | | | |
| (%) | | | | | | | | | | | | | | | |

For Formulation 3, appearance was a clear, colorless liquid under long-term storage conditions up to 6 months, regardless of TS duration. Appearance was a clear, colorless liquid under intermediate storage conditions up to 6 months for a TS cycle of 15 minutes; appearance was a clear, colorless liquid under intermediate storage conditions up to 3 months for a TS cycle of 20 and 25 minutes, with the appearance becoming clear, slightly yellow/brown liquid by 6 months. Appearance was a clear, colorless liquid under accelerated storage conditions up to 2 months, regardless of TS duration, with the appearance becoming clear, yellow/brown liquid by 3 months.

pH appeared controlled post-TS (loss of −0.3 units) from 6.9 to 6.6.

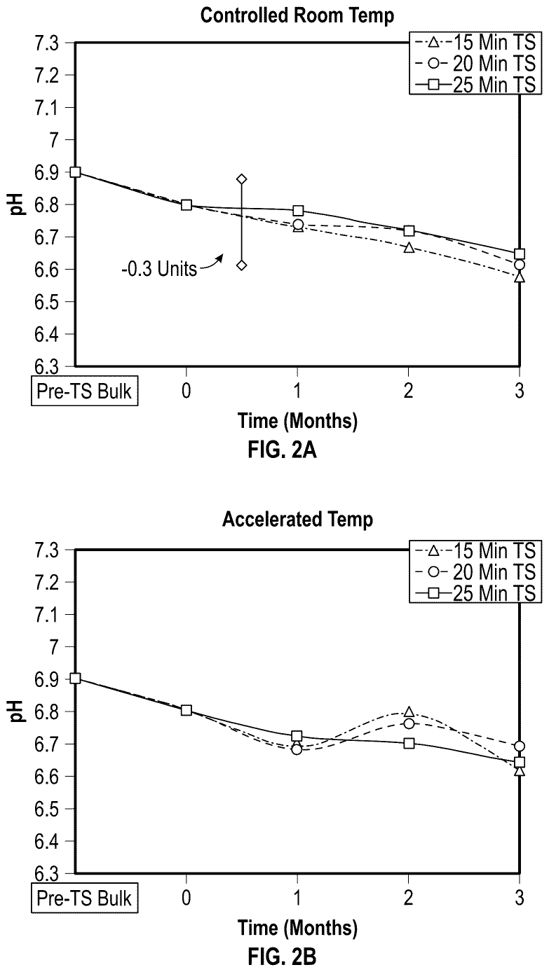

As shown in FIG. 2A, under long-term storage conditions, the pH decreased slightly over 3 months after TS regardless of TS cycle. Under intermediate storage conditions (not shown), the pH appeared to behave similarly to long-term storage conditions. As shown in FIG. 2B, under accelerated storage conditions, the pH decreased slightly over 3 months after TS regardless of TS cycle.

Color was controlled under long-term storage conditions over 2 months but under accelerated storage conditions color slightly increased starting at 1 month to 2 months.

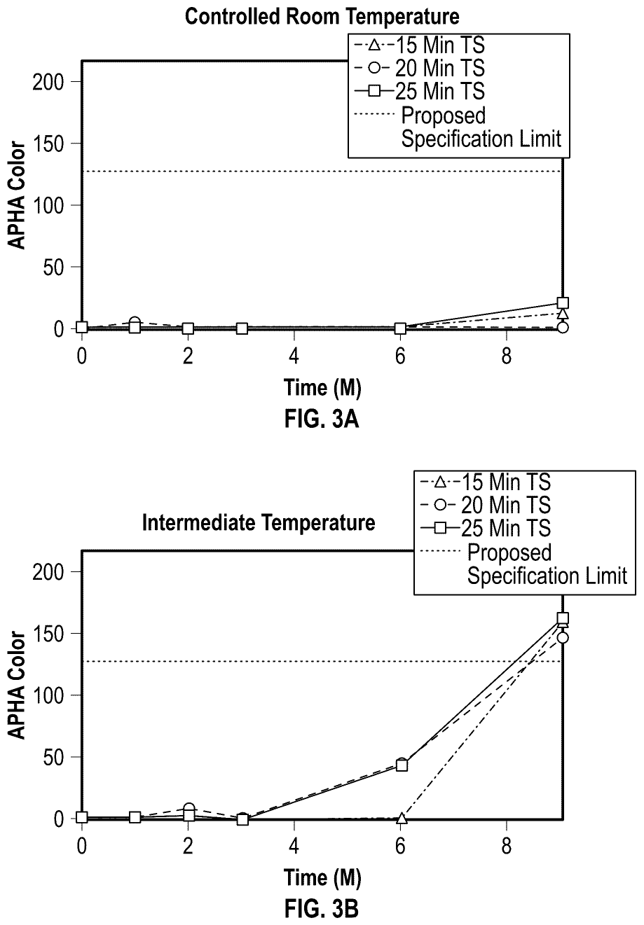

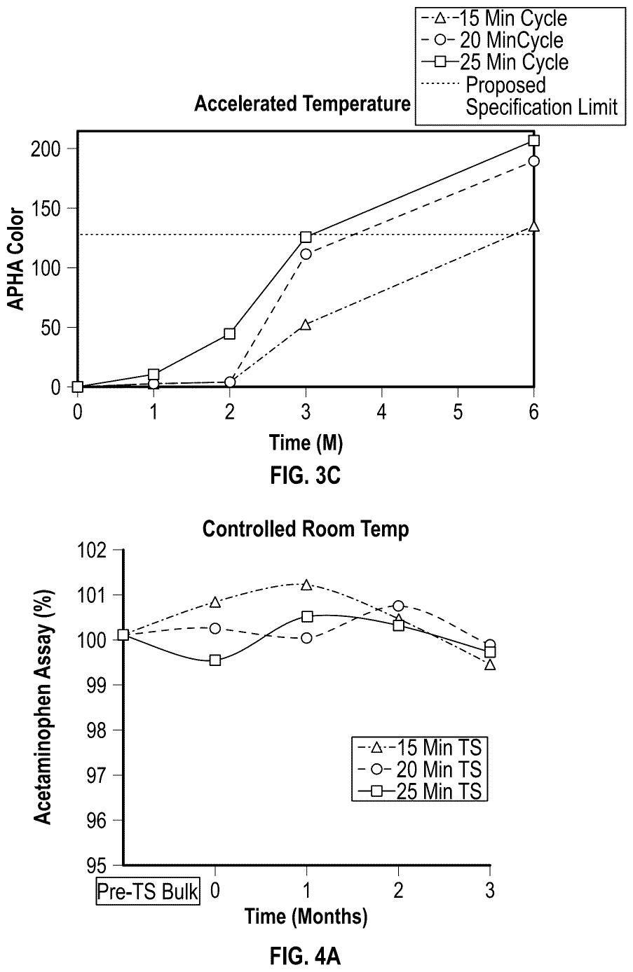

As shown in FIG. 3A and FIG. 3B, under long-term and intermediate storage conditions, respectively, the color appeared stable up to 6 months with a cycle up to 25 minutes.

As shown in FIG. 3C, under accelerated storage conditions, the color appeared to be stable from T0-2 months for a TS cycle up to 20 minutes. The color appeared to begin to increase at 1-2 months with a TS cycle time of 25 minutes. By 3 months, samples with a 20 or 25 min TS cycle were above the specification with respect to color. At 6 months, all samples were out of specification; however, the sample from the TS cycle time of 15 minutes was only just above the proposed specification.

Acetaminophen assay was stable up to 6 months under long-term, intermediate, and accelerated stability conditions. Ibuprofen appeared stable under long-term and intermediate storage conditions up to 6 months.

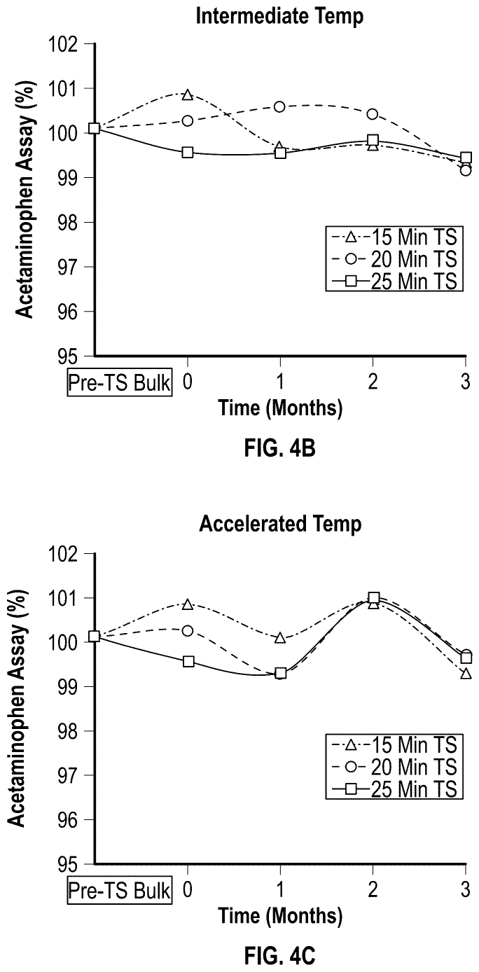

As shown in FIG. 4A, under long-term storage conditions, Formulation 3, showed minimal acetaminophen assay change with a TS cycle up to 25 minutes for up to 3 months.

As shown in FIG. 4B, under intermediate storage conditions, acetaminophen assay appeared stable with a TS cycle up to 25 minutes for up to 3 months.

As shown in FIG. 4C, under accelerated storage conditions, acetaminophen assay appeared stable with a TS cycle up to 25 minutes for up to 3 months.

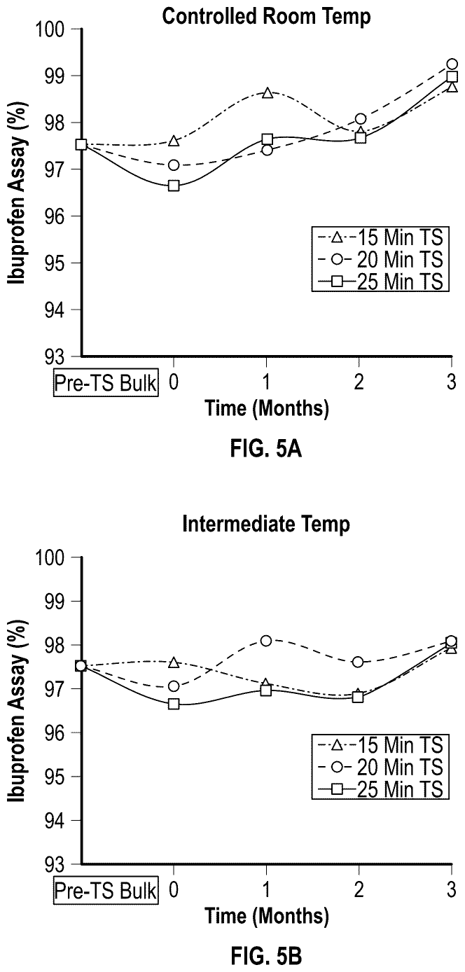

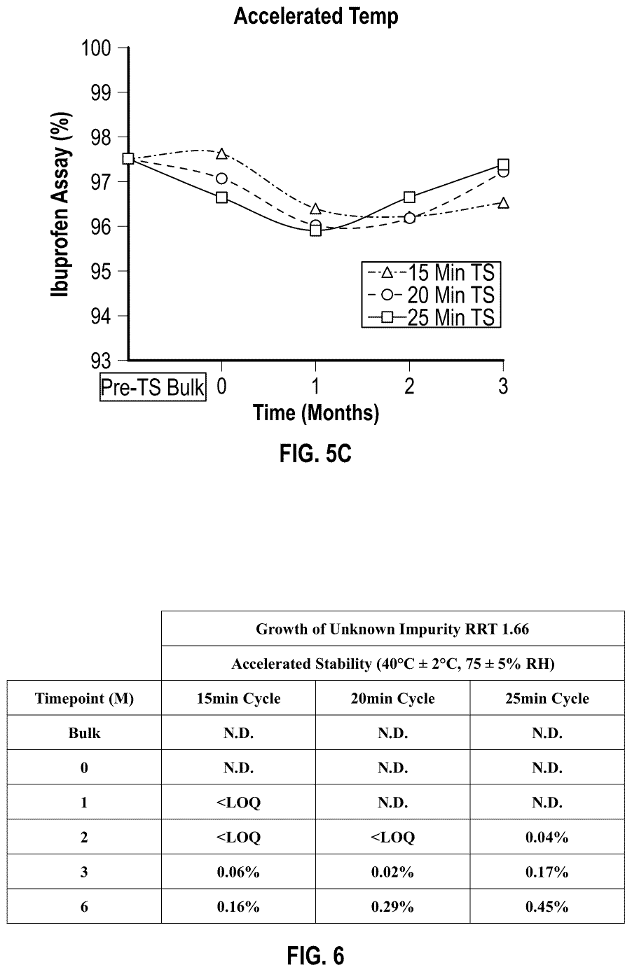

As shown in FIG. 5A, under long-term storage conditions, Formulation 3, showed minimal ibuprofen assay change with a TS cycle up to 25 minutes for up to 3 months.

As shown in FIG. 5B, under intermediate storage conditions, ibuprofen assay appeared stable with a TS cycle up to 25 minutes for up to 3 months.

As shown in FIG. 5C, under accelerated storage conditions ibuprofen assay was initially stable with a TS cycle up to 25 minutes but then shows a small decrease over a period of 1 month but is within method variability.

An unknown impurity (RRT 1.66) appeared under accelerated storage conditions, increasing after 1 month. No impurities were observed other than Unknown RRT 1.66.

FIG. 6 shows the growth of unknown impurity (RRT 1.66) under accelerated storage conditions.

Tables 15-17 show stability under long-term, intermediate, and accelerated storage conditions over time for Formulation 3 after terminal sterilization for 15, 20, and 25 minutes.

TABLE 15
(Formulation 3; TS 15 min).

| | Formulation 3-100 mL Filled Polymeric Infusion Container in Overwrap with Oxygen Scavenger - TS 15 min | | | | | | | | | | | | | | |
|---|---|---|---|---|---|---|---|---|---|---|---|---|---|---|---|
| | | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | Post-TS | 1 | 2 | 3 | 6 | 9 | 1 | 2 | 3 | 6 | 9 | 1 | 2 | 3 | 6 |
| pH | 6.8 | 6.73 | 6.67 | 6.58 | 6.53 | 6.53 | 6.73 | 6.68 | 6.52 | 6.66 | 6.60 | 6.69 | 6.79 | 6.62 | 6.75 |
| APHA Color | 1.5 | 0.7 | 1.3 | 0.5 | 1.1 | 13.7 | 1.3 | 3.0 | 0.9 | 1.1 | 159.2 | 1.1 | 3.3 | 53.9 | 136.8 |
| Acetaminophen | 100.8 | 101.2 | 100.5 | 99.5 | 99.5 | 97.9 | 99.7 | 99.7 | 99.3 | 99.5 | 98.7 | 100.1 | 100.9 | 99.3 | 99.5 |
| (%) | | | | | | | | | | | | | | | |
| Ibuprofen (%) | 101.0 | 102.0 | 101.2 | 98.7 | 99.7 | 97.1 | 100.5 | 100.2 | 97.9 | 98.5 | 96.9 | 99.7 | 99.5 | 96.5 | 97.7 |
| Sodium | 23.8 | 18.9 | 20.3 | — | 3.8 | — | 18.1 | 19.9 | — | 2.7 | — | 5.2 | 9.5 | — | 1.7 |
| Metabisulfite | | | | | | | | | | | | | | | |
| (%) | | | | | | | | | | | | | | | |

TABLE 16
(Formulation 3; TS 20 min).

| | Formulation 3-100 mL Filled Polymeric Infusion Container in Overwrap with Oxygen Scavenger - TS 20 min | | | | | | | | | | | | | | |
|---|---|---|---|---|---|---|---|---|---|---|---|---|---|---|---|
| | | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | Post-TS | 1 | 2 | 3 | 6 | 9 | 1 | 2 | 3 | 6 | 9 | 1 | 2 | 3 | 6 |
| pH | 6.8 | 6.74 | 6.72 | 6.62 | 6.56 | 6.60 | 6.76 | 6.70 | 6.56 | 6.63 | 6.66 | 6.68 | 6.76 | 6.69 | 6.71 |
| APHA Color | 1.1 | 4.5 | 1.6 | 0.7 | 0.9 | −0.2 | 1.5 | 8.8 | 1.3 | 44.7 | 146.9 | 1.3 | 3.3 | 113.7 | 190.0 |
| Acetaminophen | 100.3 | 100.0 | 100.8 | 99.9 | 99.0 | 100.1 | 100.6 | 100.4 | 99.2 | 99.4 | 100.0 | 99.3 | 101.0 | 100.1 | 99.7 |
| (%) | | | | | | | | | | | | | | | |
| Ibuprofen (%) | 100.4 | 100.8 | 101.4 | 99.2 | 99.3 | 99.2 | 101.5 | 101.0 | 98.1 | 98.8 | 98.0 | 99.3 | 99.5 | 98.2 | 97.2 |
| Sodium | 23.6 | 19.1 | 21.7 | — | 4.6 | — | 18.9 | 19.8 | — | 1.7 | — | 8.3 | 8.5 | — | 1.7 |
| Metabisulfite | | | | | | | | | | | | | | | |
| (%) | | | | | | | | | | | | | | | |

TABLE 17
(Formulation 3; TS 25 min).

| | Formulation 3-100 mL Filled Polymeric Infusion Container in Overwrap with Oxygen Scavenger - TS 25 min | | | | | | | | | | | | | | |
|---|---|---|---|---|---|---|---|---|---|---|---|---|---|---|---|
| | | 25° C. ± 2° C., 60 ± 5% RH (mo.) | | | | | 30° C. ± 2° C., 65 ± 5% RH (mo.) | | | | | 40° C. ± 2° C., 75 ± 5% RH (mo.) | | | |
| Test | Post-TS | 1 | 2 | 3 | 6 | 9 | 1 | 2 | 3 | 6 | 9 | 1 | 2 | 3 | 6 |
| pH | 6.8 | 6.78 | 6.72 | 6.65 | 6.65 | 6.54 | 6.75 | 6.70 | 6.58 | 6.64 | 6.65 | 6.72 | 6.70 | 6.64 | 6.69 |
| APHA Color | 1.3 | 0.9 | 1.1 | 1.1 | 0.1 | 20.3 | 2.2 | 3.3 | 0.9 | 40.0 | 162.2 | 1.3 | 44.7 | 127.6 | 206.3 |
| Acetaminophen | 99.6 | 100.5 | 100.3 | 99.7 | 99.6 | 99.2 | 99.5 | 99.8 | 99.4 | 99.2 | 100.1 | 99.3 | 100.9 | 99.7 | 98.5 |
| (%) | | | | | | | | | | | | | | | |
| Ibuprofen (%) | 100.0 | 101.0 | 101.1 | 99.0 | 100.4 | 98.4 | 100.3 | 100.2 | 98.0 | 98.6 | 98.2 | 99.2 | 100.0 | 97.4 | 97.2 |
| Sodium | 22.4 | 19.1 | 21.7 | — | 13.5 | — | 17.8 | 19 | — | 1.7 | — | 10.4 | 7.2 | — | 1.7 |
| Metabisulfite | | | | | | | | | | | | | | | |
| (%) | | | | | | | | | | | | | | | |

For Formulation 4, appearance was a clear, colorless liquid under long-term and intermediate storage conditions up to 3 months, regardless of TS duration. Appearance was a clear, colorless liquid under accelerated storage conditions up to 2 months, regardless of TS duration, with the appearance becoming clear, yellow/brown liquid by 3 months.

pH decreased post-TS (loss of −0.7 units) from 6.9 to 6.3. Under accelerated storage conditions, the pH dropped but then began to increase from 6.3 to 6.6.

Color slightly increased under long-term storage conditions over 2 months. Under accelerated storage conditions, color increased starting at 1 month to 2 months.

Acetaminophen assay was stable up to 2 months under long-term, intermediate, and accelerated storage conditions. Ibuprofen appeared stable under long-term and intermediate storage conditions up to 2 months however a decrease was observed at accelerated storage conditions over 2 months.

Tables 18-20 show stability under long-term, intermediate, and accelerated storage conditions over time for Formulation 4 after terminal sterilization for 15, 20, and 25 minutes.

TABLE 18
(Formulation 4; TS 15 min).

| | Formulation 4-100 mL Filled Polymeric Infusion Container in Overwrap with Oxygen Scavenger - TS 15 min | | | | | | | | | |
|---|---|---|---|---|---|---|---|---|---|---|
| | | 25° C. ± 2° C., 60 ± 5% RH (mo.) | | | 30° C. ± 2° C., 65 ± 5% RH (mo.) | | | 40° C. ± 2° C., 75 ± 5% RH (mo.) | | |
| Test | Post-TS | 1 | 2 | 3 | 1 | 2 | 3 | 1 | 2 | 3 |
| pH | 6.4 | 6.37 | 6.41 | 6.36 | 6.40 | 6.41 | 6.39 | 6.46 | 6.59 | 6.58 |
| APHA Color | 1.6 | 1.8 | 3.7 | 2.0 | 2.2 | 4.1 | 13.1 | 9.9 | 48.5 | 79.1 |
| Acetaminophen | 100.0 | 100.1 | 100.0 | 99.9 | 100.2 | 100.0 | 99.4 | 100.1 | 100.4 | 99.5 |
| (%) | | | | | | | | | | |
| Ibuprofen (%) | 100.1 | 99.8 | 99.6 | 98.0 | 99.7 | 99.2 | 97.1 | 98.8 | 98.6 | 95.9 |
| Sodium | 14.6 | 5.8 | 9.6 | — | 4.1 | 7.9 | — | 1.7 | 7.1 | — |
| Metabisulfite | | | | | | | | | | |
| (%) | | | | | | | | | | |

TABLE 19
(Formulation 4; TS 20 min).

| | Formulation 4-100 mL Filled Polymeric Infusion Container in Overwrap with Oxygen Scavenger - TS 20 min | | | | | | | | | |
|---|---|---|---|---|---|---|---|---|---|---|
| | | 25° C. ± 2° C., 60 ± 5% RH (mo.) | | | 30° C. ± 2° C., 65 ± 5% RH (mo.) | | | 40° C. ± 2° C., 75 ± 5% RH (mo.) | | |
| Test | Post-TS | 1 | 2 | 3 | 1 | 2 | 3 | 1 | 2 | 3 |
| pH | 6.4 | 6.43 | 6.36 | 6.36 | 6.37 | 6.39 | 6.38 | 6.49 | 6.57 | 6.55 |
| APHA Color | 1.6 | 1.6 | 2.8 | 1.5 | 2.4 | 4.7 | 2.6 | 4.7 | 49.6 | 68.8 |
| Acetaminophen | 99.7 | 100.7 | 100.5 | 100.3 | 100.2 | 100.3 | 100.0 | 99.9 | 102.0 | 99.8 |
| (%) | | | | | | | | | | |
| Ibuprofen (%) | 100.1 | 100.5 | 100.2 | 98.5 | 99.7 | 99.6 | 97.8 | 98.7 | 100.2 | 96.4 |
| Sodium | — | — | — | — | — | — | — | 1.9 | 7.1 | — |
| Metabisulfite | | | | | | | | | | |
| (%) | | | | | | | | | | |

TABLE 20
(Formulation 4; TS 25 min).

| | Formulation 4-100 mL Filled Polymeric Infusion Container in Overwrap with Oxygen Scavenger - TS 25 min | | | | | | | | | |
|---|---|---|---|---|---|---|---|---|---|---|
| | | 25° C. ± 2° C., 60 ± 5% RH (mo.) | | | 30° C. ± 2° C., 65 ± 5% RH (mo.) | | | 40° C. ± 2° C., 75 ± 5% RH (mo.) | | |
| Test | Post-TS | 1 | 2 | 3 | 1 | 2 | 3 | 1 | 2 | 3 |
| pH | 6.5 | 6.39 | 6.37 | 6.38 | 6.37 | 6.4 | 6.37 | 6.52 | 6.77 | 6.49 |
| APHA Color | 0.0 | 1.5 | 4.5 | 1.5 | 2.8 | 5.2 | −1.0 | 17.4 | 62.5 | 75.3 |
| Acetaminophen | 99.4 | 99.5 | 100.5 | 99.7 | 100.1 | 101.2 | 99.9 | 99.8 | 100.6 | 99.9 |
| (%) | | | | | | | | | | |
| Ibuprofen (%) | 99.2 | 99.4 | 100.0 | 97.6 | 99.6 | 100.2 | 97.1 | 98.4 | 98.1 | 96.8 |
| Sodium | 13.7 | 6.1 | 8.8 | — | 4.2 | 7.7 | — | 1.6 | 7.2 | — |
| Metabisulfite | | | | | | | | | | |
| (%) | | | | | | | | | | |

For Formulation 5, appearance was a clear, colorless liquid under long-term and intermediate storage conditions up to 3 months, regardless of TS duration. Appearance was a clear, colorless liquid under accelerated storage conditions up to 2 months, regardless of TS duration, with the appearance becoming clear, yellow/brown liquid by 3 months.

pH decreased post-TS (loss of −0.7 units) from 6.9 to 6.3. Under accelerated storage conditions, the pH dropped but then began to increase from 6.3 to 6.6.

Color slightly increased under long-term storage conditions over 2 months. Under accelerated storage conditions, color increased starting at 1 month to 2 months.

Acetaminophen assay was stable up to 2 months under long-term, intermediate, and accelerated storage conditions. Ibuprofen appeared to drop slightly under long-term storage conditions. A larger decrease was observed under intermediate storage conditions and even larger decrease (−4.4%) was observed under accelerated storage conditions over 2 months.

Tables 21-23 show stability under long-term, intermediate, and accelerated storage conditions over time for Formulation 5 after terminal sterilization for 15, 20, and 25 minutes.

TABLE 21
(Formulation 5; TS 15 min).

| | Formulation 5-100 mL Filled Polymeric Infusion Container in Overwrap with Oxygen Scavenger - TS 15 min | | | | | | | | | |
|---|---|---|---|---|---|---|---|---|---|---|
| | | 25° C. ± 2° C., 60 ± 5% RH (mo.) | | | 30° C. ± 2° C., 65 ± 5% RH (mo.) | | | 40° C. ± 2° C., 75 ± 5% RH (mo.) | | |
| Test | Post-TS | 1 | 2 | 3 | 1 | 2 | 3 | 1 | 2 | 3 |
| pH | 6.3 | 6.29 | 6.29 | 6.28 | 6.28 | 6.32 | 6.34 | 6.42 | 6.68 | 6.46 |
| APHA Color | 1.5 | −1.0 | 3.7 | 1.6 | 2.2 | 7.5 | 2.6 | 6.2 | 29.1 | 67.4 |
| Acetaminophen | 99.9 | 100.7 | 100.7 | 100.1 | 100.2 | 100.6 | 99.1 | 100.4 | 100.6 | 100.4 |
| (%) | | | | | | | | | | |
| Ibuprofen (%) | 99.1 | 99.5 | 99.0 | 96.9 | 98.5 | 98.7 | 94.8 | 97.6 | 96.8 | 95.8 |
| Sodium | 14.0 | 7.7 | 10.3 | | 4.6 | 9.6 | | 1.8 | 7.3 | |
| Metabisulfite | | | | | | | | | | |
| (%) | | | | | | | | | | |

TABLE 22
(Formulation 5; TS 20 min).

| | Formulation 5-100 mL Filled Polymeric Infusion Container in Overwrap with Oxygen Scavenger - TS 20 min | | | | | | | | | |
|---|---|---|---|---|---|---|---|---|---|---|
| | | 25° C. ± 2° C., 60 ± 5% RH (mo.) | | | 30° C. ± 2° C., 65 ± 5% RH (mo.) | | | 40° C. ± 2° C., 75 ± 5% RH (mo.) | | |
| Test | Post-TS | 1 | 2 | 3 | 1 | 2 | 3 | 1 | 2 | 3 |
| pH | 6.3 | 6.29 | 6.33 | 6.27 | 6.34 | 6.33 | 6.34 | 6.3 | 6.42 | 6.55 |
| APHA Color | 1.8 | 1.5 | 2.2 | 1.6 | 2.0 | 4.8 | 6.7 | 1.8 | 4.1 | 37.0 |
| Acetaminophen | 100.3 | 101.7 | 100.9 | 100.0 | 99.9 | 101.0 | 100.4 | 100.4 | 101.0 | 100.2 |
| (%) | | | | | | | | | | |
| Ibuprofen (%) | 99.0 | 100.0 | 99.1 | 96.8 | 98.5 | 98.7 | 96.4 | 98.0 | 97.5 | 95.4 |
| Sodium | 11.5 | 6.0 | 9.1 | | 4.8 | 8.2 | | 11.5 | 2.1 | 7.2 |
| Metabisulfite | | | | | | | | | | |
| (%) | | | | | | | | | | |

TABLE 23
(Formulation 5; TS 25 min).

| | Formulation 5-100 mL Filled Polymeric Infusion Container in Overwrap with Oxygen Scavenger - TS 25 min | | | | | | | | | |
|---|---|---|---|---|---|---|---|---|---|---|
| | | 25° C. ± 2° C., 60 ± 5% RH (mo.) | | | 30° C. ± 2° C., 65 ± 5% RH (mo.) | | | 40° C. ± 2° C., 75 ± 5% RH (mo.) | | |
| Test | Post-TS | 1 | 2 | 3 | 1 | 2 | 3 | 1 | 2 | 3 |
| pH | 6.3 | 6.26 | 6.31 | 6.27 | 6.33 | 6.32 | 6.37 | 6.40 | 6.52 | 6.48 |
| APHA Color | 2.0 | 1.5 | 3.5 | 3.2 | 0.5 | 5.2 | 4.8 | 7.7 | 3.3 | 62.2 |
| Acetaminophen | 100.7 | 100.4 | 101.2 | 98.8 | 100.2 | 100.9 | 99.8 | 99.8 | 100.6 | 100.3 |
| (%) | | | | | | | | | | |
| Ibuprofen (%) | 99.2 | 98.5 | 99.6 | 95.6 | 98.3 | 98.8 | 95.5 | 97.3 | 97.8 | 95.6 |
| Sodium | 10.2 | 5.3 | 9.5 | | 3.9 | 7.9 | | 1.8 | 8.1 | |
| Metabisulfite | | | | | | | | | | |
| (%) | | | | | | | | | | |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A liquid formulation for parenteral administration comprising:
(a) 2.8 to 3.2 mg/ml ibuprofen;
(b) 9.5 to 10.5 mg/ml acetaminophen;
(c) an isotonic agent;
(d) a buffering agent, wherein the buffering agent has a buffering capacity between pH 6 and pH 10; and
(e) an antioxidant, wherein the antioxidant comprises a sulfite compound;
wherein the liquid formulation has been terminally sterilized in a flexible polymeric infusion container, wherein terminal sterilization is characterized by an F₀ value from 5 to 15; and
wherein the liquid formulation has (a) an APHA color of 52 or less following terminal sterilization in the flexible polymeric infusion container with an F₀ value of 15 minutes and/or (b) an APHA color of 128 or less following terminal sterilization in the flexible polymeric infusion container with an F₀ value of 15 minutes and subsequent storage for up to 1 month at 25° C.±2° C. and 60% RH±5% RH.

2. The liquid formulation of claim 1, wherein the sulfite compound is present in the liquid formulation in an amount from 0.0625 to 1.0 mg/ml at the time of preparation (to) and in a reduced amount following terminal sterilization.

3. The liquid formulation of claim 1, wherein the sulfite compound is selected from the group consisting of sodium metabisulfite, sodium sulfite, and sodium bisulfite.

4. The liquid formulation of claim 1, wherein the buffering agent comprises sodium bicarbonate, sodium citrate, glycine, or L-histidine.

5. The liquid formulation of claim 1, wherein antioxidant is sodium metabisulfite and the buffering agent comprises L-histidine.

6. The liquid formulation of claim 1, wherein the ratio of acetaminophen to ibuprofen is 3.3:1 (w/w).

7. The liquid formulation of claim 1, wherein the liquid formulation has an APHA color of 128 or less following terminal sterilization and subsequent storage for up to 3, 6, or 9 months at 25° C.±2° C. and 60% RH±5% RH.

8. A flexible polymeric infusion container filled with at least 50 mL of the liquid formulation of claim 1.

9. A method for treating mild to moderate pain and/or managing moderate to severe pain, the method comprising parenteral administration of the liquid formulation of claim 1 to a subject in need thereof.

10. A pharmaceutical product comprising a stable, ready-to-use terminally sterilized liquid formulation of ibuprofen and acetaminophen packaged in a flexible polymeric infusion container, wherein the liquid formulation comprises:
(a) 2.8 to 3.2 mg/ml ibuprofen;
(b) 9.5 to 10.5 mg/ml acetaminophen;
(c) an isotonic agent;
(d) an amino acid buffering agent; and
(e) an antioxidant, wherein the antioxidant comprises a sulfite compound.

11. The pharmaceutical product of claim 10, wherein the sulfite compound is selected from the group consisting of sodium metabisulfite, sodium sulfite, and sodium bisulfite and/or the amino acid buffering agent comprises glycine or L-histidine.

12. A method for manufacturing the pharmaceutical product of claim 10, the method comprising:
providing the liquid formulation of ibuprofen and acetaminophen packaged in a flexible polymeric infusion container, wherein the liquid formulation has an initial pH of 6.0 to 7.5; and
terminally sterilizing the liquid formulation in the flexible polymeric infusion container.

13. A liquid formulation for parenteral administration comprising:
(a) 2.8 to 3.2 mg/ml ibuprofen;
(b) 9.5 to 10.5 mg/ml acetaminophen;
(c) an isotonic agent;
(d) a buffering agent; and
(e) an antioxidant, wherein the antioxidant comprises a sulfite compound;
wherein the liquid formulation has a pH of 6.0 to 7.5 at the time of preparation (to).

14. The liquid formulation of claim 13, wherein the formulation has (a) an APHA color of 52 or less following terminal sterilization in a flexible polymeric infusion container with an F₀ value of 15 minutes and/or (b) an APHA color of 128 or less following terminal sterilization in a flexible polymeric infusion container with an F₀ value of 15 minutes and subsequent storage for up to 1 month at 25° C.±2° C. and 60% RH±5% RH.

15. The liquid formulation of claim 13, wherein the ibuprofen is present as ibuprofen sodium dihydrate.

16. The liquid formulation of claim 13, wherein the sulfite compound is selected from the group consisting of sodium metabisulfite, sodium sulfite, and sodium bisulfite.

17. The liquid formulation of claim 16, wherein the sulfite compound is present in the liquid formulation in an amount from 0.0625 to 1.0 mg/ml at t₀.

18. The liquid formulation of claim 13, wherein the buffering agent comprises sodium bicarbonate, sodium citrate, glycine, or L-histidine.

19. The liquid formulation of claim 18, wherein the buffering agent is present in the liquid formulation in an amount from 0.13 to 0.26 mg/ml at t₀.

20. The liquid formulation of claim 13, wherein antioxidant is sodium metabisulfite and the buffering agent comprises L-histidine.

21. The liquid formulation of claim 13, wherein the ratio of acetaminophen to ibuprofen is 3.3:1 (w/w).

22. A flexible polymeric infusion container filled with at least 50 mL of the liquid formulation of claim 13.

23. A method for treating mild to moderate pain and/or managing moderate to severe pain, the method comprising parenteral administration of the liquid formulation of claim 13 to a subject in need thereof.

24. A liquid formulation for parenteral administration comprising:
(a) 2.8 to 3.2 mg/ml ibuprofen;
(b) 9.5 to 10.5 mg/ml acetaminophen;
(c) 20 to 50 mg/ml of an isotonic agent;
(d) 0.13 to 0.26 mg/ml of an amino acid buffering agent comprising glycine or L-histidine; and
(e) an antioxidant, wherein the antioxidant comprises a sulfite compound, wherein the sulfite compound is present in the liquid formulation in an amount from 0.0625 to 1.0 mg/ml at the time of preparation (t₀); and
wherein the liquid formulation has a pH of 6.0 to 7.5 at t₀.

25. The liquid formulation of claim 24, wherein the formulation has (a) an APHA color of 52 or less following terminal sterilization in a flexible polymeric infusion container with an F₀ value of 15 minutes and/or (b) an APHA color of 128 or less following terminal sterilization in a flexible polymeric infusion container with an F₀ value of 15 minutes and subsequent storage for up to 1 month at 25° C.±2° C. and 60% RH±5% RH.

26. The liquid formulation of claim 24, wherein the sulfite compound is selected from the group consisting of sodium metabisulfite, sodium sulfite, and sodium bisulfite.

27. The liquid formulation of claim 24, wherein the isotonic agent comprises mannitol, the antioxidant is sodium metabisulfite, and the amino acid buffering agent comprises L-histidine.

28. The liquid formulation of claim 24, wherein the ratio of acetaminophen to ibuprofen is 3.3:1 (w/w).

29. A flexible polymeric infusion container filled with at least 50 mL of the liquid formulation of claim 24.

30. A method for treating mild to moderate pain and/or managing moderate to severe pain, the method comprising parenteral administration of the liquid formulation of claim 24 to a subject in need thereof.
